(19) United States
(12) Patent Application Publication
(10) Pub. No.: US 2003/0108884 A1
(43) Pub. Date: Jun. 12, 2003

(54) Method and kit

(76) Inventors: Robert Norman Rice, Sinnamon Park (AU); Bruce Thomas Harrison, Eastern Heights (AU)

Correspondence Address: Drinker Biddle & Reath LLP, One Logan Square, 18th & Cherry Streets, Philadelphia, PA, 19103-6996, US

(21) Appl. No.: 10/081,646

(22) Filed: Feb. 22, 2002

Related U.S. Application Data
(60) Provisional application No. 60/316,308, filed on Aug. 31, 2001.

(51) Int. Cl. C12Q 1/68; G01N 33/53; C12P 19/34

(52) U.S. Cl. 435/6; 435/7.5; 435/91.2

(57) ABSTRACT

The present invention relates generally to a method for the detection of genetic expression in cells. More particularly, the present invention is directed to a method of monitoring the transcriptional activity of genetic elements including genes in a cell and more particularly to a method of determining at a quantitative, semi-quantitative or qualitative level the transcriptional activity of selected genetic elements in a cell. The present invention is further directed to a method for analyzing run-on transcription in cells and cellular organelles such as a nuclei, mitochondria and/or chloroplasts. The present invention further contemplates the use of real-time detection analysis in an amplification assay for the determination of run-on transcription in a cell and/or cellular organelles such as a nuclei, mitochondria and/or chloroplasts. The present invention further provides a kit including components of or for a kit, preferably packaged for sale with instructions for use, in the determination of the level of run-on transcription in a cell or cellular organelles such as a nuclei, mitochondria and/or chloroplasts. The method of the present invention provides, therefore, a sensitive method for the determination of genetic expression in a cell which is rapid and cost effective.

### CROSS-REFERENCE TO RELATED APPLICATION

The priority of U.S. Provisional Patent Application No. 60/316,308, filed Aug. 31, 2001, is claimed. The entire disclosure of the aforesaid provisional patent application is incorporated herein by reference.

### FIELD OF THE INVENTION

The present invention relates generally to a method for the detection of genetic expression in cells. More particularly, the present invention is directed to a method of monitoring the transcriptional activity of genetic elements including genes in a cell and more particularly to a method of determining at a quantitative, semi-quantitative or qualitative level the transcriptional activity of selected genetic elements in a cell. The present invention is further directed to a method for analyzing run-on transcription in cells and cellular organelles such as a nuclei, mitochondria and/or chloroplasts. The present invention further contemplates the use of real-time detection analysis in an amplification assay for the determination of run-on transcription in a cell and/or cellular organelles such as a nuclei, mitochondria and/or chloroplasts. The present invention further provides a kit including components of or for a kit, preferably packaged for sale with instructions for use, in the determination of the level of run-on transcription in a cell or cellular organelles such as a nuclei, mitochondria and/or chloroplasts. The method of the present invention provides, therefore, a sensitive method for the determination of genetic expression in a cell which is rapid and cost effective.

### BACKGROUND OF THE INVENTION

Reference to any prior art in this specification is not, and should not be taken as, an acknowledgment or any form of suggestion that this prior art forms part of the common general knowledge in Australia or any other country.

The physiological state of a cell, tissue or organism is characterized in part by the expression status of transcriptional units in the cell's genomic material. Generally, the transcriptional units are in the form of genes. The degree of transcriptional activation of all genes or particular groups of genes provides a fingerprint of genetic activity which alters due to a range of inter alia internal and external stimuli, physiological conditions and developmental states. The ability to regulate the process of transcription provides the molecular basis for numerous biological processes which result in or require an alteration in gene expression. Changes in a physiological state, such as during cellular differentiation or tissue specific gene expression, is frequently the result of coordinated transcriptional activation or inactivation of particular genes or groups of genes in a cell, organ or organism. Characterization of this expression status is of key importance for answering many biological questions. From a practical viewpoint, such an understanding of expression status is becoming fundamental to functional genomics and proteomics. A change in gene expression in response to a stimulus, a developmental stage, a pathological state or a physiological state, for example, is important in determining the nature and mechanism of the change in screening for agents capable of reversing a pathological condition. Patterns of gene expression are also expected to be useful in the diagnosis of pathological conditions and, for example, may provide a basis for the sub-classification of functionally different subtypes of disease conditions.

The measurement of the rate of transcription cells requires a determination of the amount of RNA generated as a transcript. A major difficulty with such assays is distinguishing between newly transcribed or nascent RNA and accumulated (mature) RNA. Generally, an RNA molecule is stable for a much longer period of time compared to the time taken for an RNA polymerase to enzymatically synthesize the same transcript. Furthermore, different RNA species have different half lives in cells, thus the steady-state level of an RNA species in a cell does not provide information about the transcriptional activity of a given gene. Thus, the signal arising from a Northern hybridization, for example, reflects the amount of accumulated RNA and does not generally provide a measurement of the transcriptional activity of the gene. Several methods have been developed to determine the rate at which nascent RNA is generated. Generally, these methods are referred to as nuclear run-on transcription assays. Because purified RNA polymerase (RNAP) will not accurately initiate transcription in vitro, run-on transcription assays in higher eukaryotes require the use of cell extracts or intact cellular organelles such as nuclei, to provide the components necessary for transcription. Cell extracts and organelles such as nuclei can be isolated in such a way that transcription of nascent RNA transcripts by RNAP is temporarily “frozen-in-time”. This is achieved by cooling intact viable cells on ice, lysing the cells and extracting the nuclei from the cellular debris. The isolated nuclei can then be resuspended in a reaction buffer containing labeled ribonucleotides. The transcription of nascent RNA transcripts is then allowed to continue by warming the nuclei to room temperature. The assay is designed to prevent the initiation of new transcripts and only those transcripts being synthesized at the time of cooling will be extended. The rate of transcription can be measured by substituting a standard ribonucleotide with a labeled ribonucleotide, typically ³²P-UTP. The newly synthesized RNA transcripts are detected by hybridization to complementary sequences present on nylon membranes or alternatively in a ribonuclease protection assay. These types of sample detection methods have a limited sensitivity and can result in the production of high background levels which can potentially result in a large signal to noise ratio which prevents the measurement of the rates of transcription.

Nuclear run-on assays detect only a very few RNA transcripts present in each nuclei and thus in order to detect run-on transcription in total nuclei, a large number of nuclei must be used. In many circumstances, the preparation of significant quantities of nuclei maybe a major limitation to the utility of a nuclear run-on assay. Furthermore, such assays require very large quantities of labeled ribonucleotides with exceedingly high specific activity. Thus, nuclear run-on experiments of this type are potentially very hazardous and can render laboratory equipment highly radioactive and unusable until the decay of the isotope reduces radioactivity to safe levels.

There is a need, therefore, for alternative methods for the detection of nascent RNA produced by the transcription of genes. There is also a need for highly sensitive quantitative methods to determine and analyze transcription of specific nucleic acid sequences. Thus, there is a need for methods to determine the level of expression of the same gene under different conditions and to provide a fingerprint of genetic expression and transcriptional activity in a cell.

The present inventors have now developed a modified nuclear run-on assay which addresses these needs for the measurement of nascent RNA transcripts resulting from the transcription. In particular, the inventors have combined real-time technology with amplification technology to produce a nuclear run-on assay which accurately determines the level of transcriptional activity within a cell.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the use of real-time protocols in combination with amplification technology to measure the level of nascent RNA associated with a transcriptional unit. In essence, the assay is a nuclear run-on assay which has been modified to measure RNA transcribed in vitro from preparations of cellular or viral nucleic acid or organelles such as nuclei, mitochondria or chloroplasts by protocols involving measurement of the rate of accumulation of product. Preferably, this is conducted by real-time reverse transcriptase (RT)-PCR. The modified method permits a genetic fingerprint of expression status to be formulated for a cell by determining transcriptional activity. The modified assay is useful inter alia in screening the effects of genetic and small molecule agents on gene expression and monitoring changes in gene expression in response to internal and external stimuli, pathological conditions or changes in developmental stages.

Accordingly, one aspect of present invention contemplates a method for determining the activity of a transcriptional unit or a plurality of transcriptional units in a cell, said method comprising obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

Another aspect of the present invention provides a method for determining changes in activity of a transcriptional unit or plurality of transcriptional units in a cell or cell linage, said method comprising obtaining a preparation comprising said transcriptional units comprising nascent RNA strands attached thereto before or after exposure of said cell to internal or external stimulus or at different developmental stages of said cell or cell lineages under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

A further aspect of the present invention contemplates an assay device in the form of a kit useful in determining the activity of a transcriptional unit or plurality of transcriptional units in a cell, said kit comprising in compartmental form multiple compartments each adapted to comprise one or more of buffers, diluents and enzymes in single or multiple components which are required to be admixed prior to use, said kit farther comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

Another aspect of the present invention contemplates an assay device in the form of a kit useful in determining the activity of a transcriptional unit or plurality of transcriptional units in a cell, said kit comprising in compartmental form multiple compartments each adapted to comprise one or more of buffers, diluents and enzymes in single or multiple components which are required to be admixed prior to use, said kit further comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and amplification means via real-time PCR to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

A further aspect of the present invention contemplates an assay device in the form of a kit useful in determining the activity of a transcriptional unit or plurality of transcriptional units in a cell, said kit comprising in compartmental form multiple compartments each adapted to comprise one or more of buffers, diluents and enzymes in single or multiple components which are required to be admixed prior to use, said kit further comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means via binding of a biotin label on the RNA transcripts to an immobilized molecule capable of binding to biotin to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

### BRIEF DESCRIPTION OF THE FIGURES

FIG. 1a is a graphical and tabular representation of amplification plots and quantitation data for human BRN2 (duplexed with human GAPDH—FIG. 1b).

FIG. 1b is a graphical and tabular representation of amplification plots and quantitation data for human GAPDH (duplexed with human BRN2—FIG. 1a).

FIG. 2a is a graphical and tabular representation of amplification plots and quantitation data for murine B16 tyrosinase (duplexed with murine GAPDH—FIG. 2b).

FIG. 2b is a graphical and tabular representation of amplification plots and quantitation data for GAPDH (duplexed with murine B16 tyrosinase —FIG. 2a).

FIG. 3a is a graphical and tabular representation of amplification plots and quantitation data for EGFP (duplexed with murine GAPDH—FIG. 3b).

FIG. 3b is a graphical and tabular representation of amplification plots and quantitation data for murine GAPDH (duplexed with EGFP—FIG. 3a).

FIG. 4a is a graphical and tabular representation of amplification plots and quantitation data for EGFP (duplexed with human GAPDH—FIG. 4b).

FIG. 4b is a graphical and tabular representation of amplification plots and quantitation data for human GAPDH (duplexed with EGFP—FIG. 4a).

FIG. 5ais a graphical and tabular representation of amplification plots and quantitation data for human endogenous HER2 (duplexed with human GAPDH—FIG. 5b).

FIG. 5b is a graphical and tabular representation of amplification plots and quantitation data for human GAPDH (duplexed with human endogenous HER2—FIG. 5a).

FIG. 6a is a graphical and tabular representation of amplification plots and quantitation data for HER-2 exogenous assay (duplexed with human GAPDH—FIG. 6b) which exemplifies the linearity of the standard curves of the duplexed real-time RT-PCR method on a DNA template.

FIG. 6a is a graphical and tabular representation of amplification plots and quantitation data for human GAPDH (duplexed with HER-2 exogenous assay—FIG. 6b) which exemplifies the linearity of the standard curves of the duplexed real-time RT- PCR method on a DNA template.

### DETAILED DESCRIPTION OF THE PREFERRED EMBODIMENTS

The present invention is predicated in part on the finding that nascent RNA transcripts extended in the presence of a labeled ribonucleotide have utility as efficient templates for amplification reactions. This provides a finger print of genetic activity in a cell such as in response to internal or external stimuli as well as resulting from different physiological or developmental states. Consequently, this finding leads to an improved method for the quantitative or qualitative detection of transcriptional activity in cells by combining amplification methodologies with real-time analysis techniques. The subject method enables the determination of transcriptional activity in a cell not possible through sampling techniques which can only determine a level of transcription at a static point in time.

Transcription will be understood as the process by which an RNA molecule is produced from a nucleic acid template. A nucleic acid template may be RNA or DNA. An RNA transcript is regarded as any RNA molecule which is synthesized by an enzymatic process and/or a series of chemical reactions. A “nascent RNA molecule” should be understood as the portion of an RNA transcript associated with a transcriptional unit or gene. The term “nascent” is used to highlight the juvenile nature of the transcript relative to a complete or mature transcript. However, the term “mRNA” or “transcript” is to be understood as encompassing a nascent RNA molecule. A nascent transcript is generally one which is capable of extension in a run-on transcription.

Accordingly, the present invention contemplates a method for determining the activity of a transcriptional unit or a plurality of transcriptional units in a cell, said method comprising obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

Reference to “determining the activity” includes a quantitative or qualitative determination as to the level of nascent RNA associated with a transcriptional unit. The higher the activity, the more transcription of a particular transcriptional unit was taking place at the time of generation of the preparation.

A “transcriptional unit” refers to genetic material which, in a cell, is capable of acting as a template for generating a transcript through the process of transcription. A transcriptional unit may be naturally occurring or generated by, for example, recombinant means. A gene is regarded as an example of a transcriptional unit.

The cell may be a prokaryotic or eukaryotic cell. As prokaryotes do not have nuclei as such, a preparation comprising nuclear material including nascent RNAs is prepared. The present method further enables the detection of viral RNA transcripts in a cell.

A prokaryotic microorganism includes bacteria such as Gram positive, Gram negative and Gram variable bacteria and intracellular bacteria. Examples of bacteria contemplated herein include the speices of the genera Treponema, Borrelia, Neisseria, Legionella, Bordetella, Escherichia, Salmonella, Shigella, Klebsiella, Yersinia, Vibrio, Hemophilus, Rickettsia, Chlamydia, Mycoplasma, Staphylococcus, Streptococcus, Bacillus, Clostridium, Corynebacterium, Pseudomonas, Proprionibacterium, Mycobacterium, Ureaplasma and Listeria.

Particularly preferred species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsi, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfiingens, Corynebacterium diphtheriae, Proprionibacterium acnes, Mycobacterium tuberculosis, Mycobacterium leprae, Listeria monocytogenes, Pseudomonas aeruginosa and Pseudomonas putida.

A eukaryotic cell includes a yeast or fungus such as but not limited to Microsporidium, Pneumocystis carinii, Candida albicans, Aspergillus, Histoplasma capsulatum, Blastomyces dermatitidis, Cryptococcus neoformans, Trichophyton and Microsporum. The cells may also be from worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania. The eukaryotic cells may also be from mammals such as humans, primates, livestock animals, companion animals and laboratory test animals.

Viruses contemplated herein include HIV, hepatitis virus (e.g. Hep A, Hep B, Hep C and non-A, non-B Hep virus), adenoviruses, papovaviruses, herpes viruses: simplex, varicella-zoster, Epstein-Barr, CMV, pox viruses: smallpox, vaccinia, rhinoviruses, polio virus, rubella virus, arboviruses, rabies virus, influenza viruses A and B, measles virus, mumps virus and HTLV I and II.

A “plurality of transcriptional units” may comprise a group of separately transcribable units or may comprise multiple genes transcribed as a single polycistronic message. “Conditions sufficient to temporarily inhibit or substantially reduce continued transcription” means any chemical or physical intervention which inhibits transcription of a transcriptional unit. Such conditions include temperature and chemical inhibitors. The use of chemical inhibitors requires that the inhibition be reversible. The use of temperature is preferred such as placing the preparation on ice or under conditions to rapidly reduce the temperature to a level to inhibit or otherwise reduce transcription of the transcriptional units. Preferred temperature conditions for blocking transcription including from about 0° C. to about 10° C. and more preferably from about 0° C. to about 4° C.

The subject method is particularly useful in assessing transcriptional activity in a cell in response to different internal or external stimuli or in different developmental states or stages. For example, transcriptional activity may be determined in the presence or absence of introduced genetic molecules such as co-suppression or antisense molecules or RNAi-inducing molecules or in the presence or absence of introduced proteinaceous or non-proteinaceous molecules. Examples of the latter are DNA/RNA-binding proteins, chemicals from a chemical library or molecules identified from natural product screening such as from coral, sea life associated in the coral, plants, soil or various aqueous environments. Furthermore, the transcriptional activity of cells at various points in their development may also be assessed such as comprising undifferentiated stem cells to differential stem cells or committed lineage cells.

Accordingly, another aspect of the present invention provides a method for determining changes in activity of a transcriptional unit or plurality of transcriptional units in a cell or cell linage, said method comprising obtaining a preparation comprising said transcriptional units comprising nascent RNA strands attached thereto before or after exposure of said cell to internal or external stimulus or at different developmental stages of said cell or cell lineages under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

The expression “internal or external stimuli” includes the effects of co-suppression molecules, anti-sense molecules, RNAi-inducing molecules as well as proteinaceous and non-proteinaceous molecules. Preferred antisense molecules are from about 10 base pairs long to about 2000 base pairs long but more preferably from about 12 to about 30 base pairs long such as 13, 18 and 22 base pairs in length.

Preferred co-suppression molecules include double-stranded RNA molecules forming a hairpin with or without single-stranded portions in the form of a “bulge” or “bubble”.

The present invention combines detection using real-time analysis and optionally with amplification methodologies. Amplification methodologies contemplated herein include the polymerase chain reaction (PCR) such as disclosed in U.S. Pat. Nos. 4,683,202 and 4,683,195 (Mullis); the ligase chain reaction (LCR) such as disclosed in European Patent Application No. EP-A-320 308 (Backman et al.) and gap filling LCR (GLCR) or variations thereof such as disclosed in International Patent Publication No. WO 90/01069 (Segev), European Patent Application EP-A-439 182 (Backman et al.), British Patent No. GB 2,225,112A (Newton et al.) and International Patent Publication No. WO 93/00447 (Birkenmeyer et al.). Other amplification techniques include Qβ replicase such as described in the literature; Stand Displacment Amplification (SDA) such as described in European Patent Application Nos. EP-A-497 272 (Walker) and EP-A-500 224 (Walker et al.) and Walker et al. (1992); Self-Sustained Sequence Replication (3SR) such as described in Fahy et al. (1991) and Nucleic Acid Sequence-Based Amplification (NASBA) such as described in the literature.

Some amplification reactions, for example, PCR and LCR, involve cycles of alternately high and low set temperatures, a process known as “thermal cycling”. PCR or “polymerase chain reaction” is an amplification reaction in which a polymerase enzyme, usually thermostable, generates multiple copies of the original sequence by extension of a primer using the original nucleic acid as a template. PCR is described in more detail in U.S. Pat. Nos. 4,683,202 and 4,683,195. LCR or “ligase chain reaction” is a nucleic acid amplification reaction in which a ligase enzyme, usually thermostable, generates multiple copies of the original sequence by ligating two or more oligonucleotide probes while they are hybridized to the target. LCR and its variation, Gap LCR, are described in more detail in European Patent Application Nos. EP-A-320-308 (Backman et al.) and EP-A-439-182 (Backman et al.) and International Patent Publication No. WO 90/100447 (Birkenmeyer et al.) and elsewhere.

The PCR amplification process is the most preferred in practicing the present invention.

Accordingly, another aspect of the present invention contemplates an assay device in the form of a kit useful in determining the activity of a transcriptional unit or plurality of transcriptional units in a cell, said kit comprising in compartmental form multiple compartments each adapted to comprise one or more of buffers, diluents and enzymes in single or multiple components which are required to be admixed prior to use, said kit further comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and amplification means via real-time PCR to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

Real-time analysis technologies permit accurate and specific amplification products (e.g. PCR products) to be quantitatively detected within an amplification vessel during the exponential phase of the amplification process, before reagents are exhausted and the reaction plateau's or non-specific amplification limits the reaction. The particular cycle of amplification at which the detected amplification signal first crosses a set threshold is proportional to the starting copy number of the target molecules.

Instruments capable of measuring real-time including Taq Man 7700 AB (Applied Biosystems), Rotorgene 2000 (Corbett Research), LightCycler (Roche), iCycler (Biorad) and Mx4000 (Stratagene).

The method of the present invention is suitable for use with a number of direct reaction detection technologies and chemistries such as Taq Man (Perkin-Elmer), molecular beacons and the LightCycler (trademark) fluorescent hybridization probe analysis (Roche Molecular Systems).

One useful system for real-time DNA amplification and detection is the LightCyler (trademark) fluorescent hybridization probe analysis. This system involves the use of three essential components: two different oligonucleotides (labeled) and the amplification product. Oligonucleotide 1 carries a fluorescein label at its 3′ end whereas oligonucleotid 2 carries another label, LC Red 640 or LC Red 705, at its 5′ end. The sequence of the two oligonucleotides are selected such that they hybridize to the amplified DNA fragment in a head to tail arrangement. When the oligonucleotides hybridize in this orientation, the two fluorescence dyes are position in close proximity to each other. The first dye (fluorescein) is excited by the LightCyler's LED (Light Emitting Diode) filtered light source and emits green fluorescent light at a slightly longer wavelength. When the two dyes are in close proximity, the emitted energy excites the LC Red 640 or LC Red 705 attached to the second hybridization probe that subsequently emits red fluorescent light at an even longer wavelength. This energy transfer, referred to as FRET (Forster Resonance Energy Transfer or Fluorescence Resonance Energy Transfer) is highly dependent on the spacing between the two dye molecules. Only if the molecules are in close proximity (a distance between 1-5 nucleotides) is the energy transferred at high efficiency. Choosing the appropriate detection channel, the intensity of the light emitted by the LC Red 640 or LC Red 705 is filtered and measured by optics in the thermocycler. The increasing amount of measured fluorescence is proportional to the increasing amount of DNA generated during the ongoing PCR process. Since LC Red 604 and LC Red 705 only emits a signal when both oligonucleotides are hybridized, the fluorescence measurement is performed after the annealing step. Using hybridization probes can also be beneficial if samples containing very few template molecules are to be examined. DNA-quantification with hybridization probes is not only sensitive but also highly specific. It can be compared with agarose gel electrophoresis combined with Southern blot analysis but without all the time consuming steps which are required for the conventional analysis.

The “Taq Man” fluorescence energy transfer assay uses a nucleic acid probe complementary to an internal segment of the target DNA. The probe is labelled with two fluorescent moieties with the property that the emission spectrum of one overlaps the excitation spectrum of the other; as a result, the emission of the first fluorophore is largely quenched by the second. The probe if present during PCR and if PCR product is made, the probe becomes susceptible to degradation via a 5′-nuclease activity of Taq polymerase that is specific for DNA hybridized to template. Nucleolytic degradation of the probe allows the two fluorophores to separate in solution which reduces the quenching and increases intensity of emitted light.

Probes used as molecular beacons are based on the principle of single-stranded nucleic acid molecules that possess a stem-and-loop structure. The loop portion of the molecule is a probe sequence that is complementary to a predetermined sequence in a target nucleic acid. The stem is formed by the annealing of two complementary arm sequences that are on either side of the probe sequence. The arm sequences are unrelated to the target sequence. A fluorescent moiety is attached to the end of one arm and a non-fluorescent quenching moiety is attached to the end of the other arm. The stem keeps these two moieties in close proximity to each other caqusing the fluorescence of the fluorophore to be quenched by fluorescence resonance energy transfer. The nature of the fluorophore-quencher pair that is preferred is such that energy received by the fluorophore is transferred to the quencher and dissipated as heat rather than being emitted as light. As a result, the fluorophore is unable to fluoresce. When the probe encounters a target molecule, it forms a hybrid that is no longer and more stable than the hybrid formed by the arm sequences. Since nucleic acid double helices are relatively rigid, formation of a probe-target hybrid precludes the simultaneous existence of a hybrid formed by the arm sequences. Thus, the probe undergoes a spontaneous conformational change that forces the arm sequences apart and causes the fluorophore and quencher to move away from each other. Since the fluorophore is no longer in close proximity to the quencher, it fluoresces when illuminated by an appropriate light source. The probes are term “molecular beacons” because they emit a fluorescent signal only when hybridized to target molecules.

SYBR (registered trademark) is also useful. SYBR is a fluorescent dye which may be rills used in ABI sequence detection systems such as ABI PRISM 770 (registered trademark), Rotorgene 2000 (Corbett Research), Mx4000 (Stratagene), GeneAmp 5700, LightCycler (registered trademark) and iCycler (trademark).

A number of real-time fluorescent detection thermocyclers are currently available with the chemistries being interchangeable with those discussed above as the final product is emitted fluorescence. Such thermocyclers include the Perkin Elmer Biosystems 7700, Corbett Research's Rotorgene, the Hoffman La Roche LightCycler, the Stratagene Mx4000 and the Biorad iCycler. It is envisaged that any of the above theremocyclers could be adapted to accommodate the method of the present invention.

Exemplary fluorophores include but are not limited to 4-acetamido-4′-isothiocyanatostilbene-2,2′disulfonic acid acridine and derivatives including acridine, acridine isothiocyanate, 5-(2′-aminoethyl)aminonaphthalene-1-sulfonic acid (EDANS), 4-amino-N-[3-vinylsulfonyl)-phenyl]naphthalimide-3,5 disulfonate (Lucifer Yellow VS) anthranilamide, Brilliant Yellow, coumarin and derivatives including coumarin, 7-amino-4-methylcoumarin (AMC, Coumarin 120), 7-amino-4-trifluoromethylcoumarin (Coumarin 151), Cy3, Cy5, cyanosine, 4′,6-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL) 4-dimethylaminophenyl-azophenyl-4′-isothiocyanate (DABITC), eosin and derivatives including eosin, eosin isothiocyanate, erythrosin and derivatives including erythrosin B, erythrosin isothiocyanate, ethidium, fluorescein and derivatives including 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate, QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene and derivatives including, pyrene, pyrene butyrate, succinimidyl 1-pyrene butyrate, Reactive Red 4 (Cibacron [registered trademark] Brilliant Red 3B-A), rhodamine and derivatives, 6-carboxy-X-rhodamine (ROX), 6-carboxyrhodamine (R6G), lissamine rhodamine B sulfonyl chloride, rhodamine (Rhod), rhodamine B, rhodamine 110, rhodamine 123, rhodamine X isothiocyanate, sulforhodamine B, sulforhodamine 101, sulfonyl chloride derivative of sulforhodamine 101 (Texas Red), N,N,N′N′-tetramethyl-6-carboxyrhodamine (TAMRA), tetramethyl rhodamine, tetramethyl rhodamine isothiocyanate (TRITC), riboflavin, rosolic acid, terbium chelate derivatives.

The present invention permits the use of a range of capture and immobilization methodologies to capture the nascent RNA transcripts. Dynabead technology is the most convenient up to the present time. In one example, biotin or a related molecule is incorporated into an RNA molecule and this permits immobilization to a bead coated with a biotin ligand. Examples of such ligands include streptavidin, avidin and anti-biotin antibodies.

Furthermore, the present invention further proposes to modify Dynabead immobilization to enable labeled transcripts to be cleaved or eluted off the bead by the incorporation of a cleavable or otherwise labile linker between, for example, a UTP and a biotin label or between a Dyabead and streptavidin. One preferred cleavable marker is a disulfide bridge which could be disrupted by dithiothreiol (DTT) or other reducing agent. DTT is particularly useful as it is compatible with Taq Man chemistry. The release of, for example, biotin UTP-labeled transcripts from a streptavidin Dynabead farther enables a more homogenous sample and reaction as the dense Dynabeads tend to sink rapidly and form a pellet during the steps of the process which has the potential of causing inaccurate aliquoting of the sample and poor access of RT and PCR reagents to nasent mRNA transcripts and cDNA.

A “nucleic acid” as used herein, is a covalently linked sequence of nucleotides in which the 3′ position of the pentose of one nucleotide is joined by a phosphodiester group to the 5′ position of the pentose of the next nucleotide and in which the nucleotide residues (bases) are linked in specific sequence; i.e. a linear order of nucleotides. A “polynucleotide” as used herein, is a nucleic acid containing a sequence that is greater than about 100 nucleotides in length. An “oligonucleotide” as used herein, is a short polynucleotide or a portion of a polynucleotide. An oligonucleotide typically contains a sequence of about two to about one hundred bases. The word “oligo” is sometimes used in place of the word “oligonucleotide”.

“Nucleoside”, as used herein, refers to a compound consisting of a purine [guanine (G) or adenine (A)] or pyrimidine [thymine (T), uridine (U) or cytidine (C)] base covalently linked to a pentose, whereas “nucleotide” refers to a nucleoside phosphorylated at one of its pentose hydroxyl groups. “XTP”, “XDP” and “XMP” are generic designations for ribonucleotides and deoxyribonucleotides, wherein the “TP” stands for triphosphate, “DP” stands for diphosphate, and “IMP” stands for monophosphate, in conformity with standard usage in the art. Subgeneric designations for ribonucleotides are “NMP”, “NDP” or “NTP”, and subgeneric designations for deoxyribonucleotides are “dNMP”, “dNMP” or “dNTP”. Also included as “nucleoside”, as used herein, are materials that are commonly used as substitutes for the nucleosides above such as modified forms of these bases (e.g. methyl guanine) or synthetic materials well known in such uses in the art, such as inosine.

As used herein, the term “nucleic acid probe” refers to an oligonucleotide or polynucleotide that is capable of hybridizing to another nucleic acid of interest under low stringency conditions. A nucleic acid probe may occur naturally as in a purified restriction digest or be produced synthetically, by recombinant means or by PCR amplification. As used herein, the term “nucleic acid probe” refers to the oligonucleotide or polynucleotide used in a method of the present invention. That same oligonucleotide could also be used, for example, in a PCR method as a primer for polymerization, but as used herein, that oligonucleotide would then be referred to as a “primer”. In some embodiments herein, oligonucleotides or polynucleotides contain a modified linkage such as a phosphorothioate bond.

As used herein, the terms “complementary” or “complementarity” are used in reference to nucleic acids (i.e. a sequence of nucleotides) related by the well-known base-pairing rules that A pairs with T and C pairs with G. For example, the sequence 5′-A-G-T-3′, is complementary to the sequence 3′-T-C-A-5′. Complementarity can be “partial” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the target nucleic acid sequence under conditions of low stringency as described below or, preferably, in polymerase reaction buffer (Promega, M195A) heated to 95° C. and then cooled to room temperature. As used herein, when the nucleic acid probe is referred to as partially or totally complementary to the target nucleic acid, that refers to the 3′-terminal region of the probe (i.e. within about 10 nucleotides of the 3′-terminal nucleotide position).

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, 1962). However, the Tₘ of a duplex DNA decreases by 1° C. with every increase of 1% in the number of mismatch base pairs (Bonner and Laskey, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The present invention also contemplates kits for determining the activity of a transcription unit in a cell. The kits may comprise many different forms but in a preferred embodiment, the kits are designed for analysis by mass spectrometry, fluorescence spectroscopy (e.g. Syber Green) or absorption spectroscopy.

The kit may also comprise instructions for use.

Accordingly, another aspect of the present invention contemplates an assay device in the form of a kit useful in determining the activity of a transcriptional unit or plurality of transcriptional units in a cell, said kit comprising in compartmental form multiple compartments each adapted to comprise one or more of buffers, diluents and enyzmes in single or multiple components which are required to be admixed prior to use, said kit further comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

A particularly useful kit comprises one or more buffers, diluents and enzymes. Particularly useful buffers are described in the Examples and include storage buffers, cell lysis buffers and buffers for use in amplification reactions. Enzymes include polymerases. The kit may also comprise a series of labeled or unlabeled deoxyribonucleotides and/or ribonucleotides.

Conveniently, the kits are adapted to contain compartments for two or more of the above-listed components. Furthermore, buffers, nucleotides and/or enzymes may be combined into a single compartment.

One form of kit contemplated herein optionally further comprises at least one nucleic acid probe which is complementary to a nucleic acid target sequence and comprising a fluorophore. The nucleic acid probe may also include at least one label. The nucleic acid probe may also comprise a nucleotide analogue.

The Taq polymerase is an example of a suitable DNA polymerase which is thermostable. The thermostable DNA polymerase is used in an amount sufficient for a hybridized probe to release an identifier nucleotide. This amount may vary with the enzyme used and also with the temperature at which depolymerization is carried out. An enzyme of a kit is typically present in an amount sufficient to permit the use of about 0.1 to 100 U/reaction; in particularly preferred embodiments, the concentration is about 0.5 U/reaction.

As stated above, instructions optionally present in such kits instruct the user on how to use the components of the kit to perform the various methods of the present invention. It is contemplated that these instructions include a description of the detection methods of the subject invention, including detection by mass spectrometry, fluorescence spectroscopy and absorbance spectroscopy.

The present invention further contemplates kits which contain a nucleic acid probe for a nucleic acid target of interest with the nucleic acid probe being complementary to a predetermined nucleic acid target and comprising an identifier nucleotide. In another embodiment, the kit contains multiple probes, each of which contain a different base at an interrogation position or which are designed to interrogate different target DNA sequences. In a contemplated embodiment, multiple probes are provided for a set of nucleic acid target sequences that give rise to analytical results which are distinguishable for the various probes.

It is contemplated that a kit contains a vessel containing a purified and isolated enzyme whose activity is to release one or more nucleotides from the 3′ terminus of a hybridized nucleic acid probe and a vessel containing pyrophosphate. In one embodiment, these items are combined in a single vessel. It is contemplated that the enzyme is either in solution or provided as a solid (e.g. as a lyophilized powder), the same is true for the pyrophosphate. Preferably, the enzyme is provided in solution. Some contemplated kits contain labeled nucleic acid probes. Other contemplated kits further comprise vessels containing labels and vessels containing reagents for attaching the labels. Microtiter trays are particularly useful and these may comprise from two to 100,000 wells or from about six to about 10,000 wells or from about six to about 1,000 wells.

As discussed above, the nucleic acid probe optionally comprises a label, or a nucleotide analog. Thus, in some embodiments of a kit or composition, the identifier nucleotide comprises a fluorescent label and the probe optionally further comprises a fluorescence quencher or enhancer. As mentioned above, exemplary useful fluorophores are Fluorescein, 5-carboxyfluorescein (FAM), 2′7′ dimethoxy-4′5′-dichloro-6-carboxy-fluorescein (JOI), rhodamine, 6-carboxyrhodamine (R6G), N,N,N,N-tetramethyl-6-carboxyrhodamine (TAMRA), 6-carboxy-X-rhodamine (ROX), 4-(4′-dimethylamino-phenylazo)benzoic acid (DABCYL) and 5-(2′-aminoethyl)aminonaphthalene-1-sulfonic acid (EDANS). In other embodiments of a kit or composition, the identifier nucleotide comprises a non-natural nucleotide analog.

“Purification and isolation” when used in relation to a nucleic acid or protein, refers to a process by which a nucleic acid sequence or protein is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature. Methods for the purification of a molecule are well known in the art, and include but are not limited to chromatography, electrophoresis, HPLC, reverse phase liquid chromatography immunoadsorbtion and immunoprecipitation, ion exchange chromatography, affinity matrix chromatography and immoblized metal ion affinity chromatography.

The present invention is particularly directed to the purification and isolation of biotin labeled nascent RNA transcripts and further contemplates a method of purifying nascent RNA transcripts containing one or more biotin labeled ribonucleotides.

In one preferred embodiment of the present invention, purification and isolation of the biotin labeled nascent RNA transcript is achieved by binding to a solid matrix. As used herein, the term “solid matrix” refers to a material in a solid form to which a biotin labeled nascent transcript can be attached. Examples of a solid matrix include a magnetic particle, or a magnetic glass particle, a polymeric microsphere, a filter material, and include polymers surfaces such as those on the surface of a micro-titre plate or capillary tube or cylinder. Preferably, the solid matrix is capable of being coated with a compound that is capable of binding biotin. In one aspect of the present invention, the compound that is capable of binding biotin is streptavidin. In a preferred embodiment the solid matrix is coated with streptavidin.

In a particularly preferred embodiment, the solid matrix is a Dynabead.

The streptavidin may be covalently bonded to the solid matrix or may interact with the matrix via electrostatic interactions. Without intending to limit the scope of the invention, it is preferred that the sold matrix is a magnetic particle coated with streptavidin. Although magnetic beads coated with streptavidin are contemplated in the subject invention, the present invention also contemplates the use of solid surfaces such as hereinbefore mentioned, in combination with biotin binding compounds and molecules other than streptavidin that are capable of binding biotin such as avidin or anti-biotin antibodies.

Accordingly, the present invention further contemplates an assay device in the form of a kit useful in determining the activity of a transcriptional unit or plurality of transcriptional units in a cell, said kit comprising in compartmental form multiple compartments each adapted to comprise one or more of buffers, diluents and enyzmes in single or multiple components which are required to be admixed prior to use, said kit further comprising instructions for use wherein the method is conducted by obtaining a preparation comprising said transcriptional units comprising nascent RNA strands attached thereto from said cell under conditions sufficient to temporarily inhibit or substantially reduce continued transcription and then placing said transcriptional units under conditions to permit transcription in the presence of labeled ribonucleotides to thereby provide a population of transcripts including nascent transcripts comprising one or more of said labeled ribonucleotides and subjecting said population of transcripts including nascent RNA molecules to isolation and purification means via binding of a biotin label on the RNA transcripts to immobilized streptavidin to generate a purified population of transcripts and simultaneously or sequentially subjecting said population of transcripts including nascent RNA molecules comprising one or more labeled ribonucleotides to detection and optionally including amplification means to measure the appearance of a detectable product wherein the rate of appearance of product is proportional to the amount of transcript including nascent RNA molecules associated with a particular transcriptional unit isolated from said cell which in turn determines the transcriptional activity of said transcriptional unit or plurality of transcriptional units.

Molecules other than streptavidin, that are capable of binding biotin include but are not limited to in vitro evolved molecules such as in vitro evolved DNA and RNA molecules. Examples of molecules capable of binding biotin other than avidin and antibodies include biotin binding aptamer molecules such as RNA and DNA aptamer molecules. Such molecules are well known in the art. The biotin binding molecule may be covalently linked to the solid matrix, or may interact with it via electrostatic interaction. In this embodiment, the nascent transcript comprising a biotin labeled nucleotide can be bound to the surface of a solid matrix that is coated with an in vitro evolved RNA or DNA aptamer molecules. As well as RNA and DNA molecules that bind biotin, proteins other than streptavidin are contemplated by the present invention. Non-streptavidin proteins capable of binding biotin include but are not limited to, biotin carboxyl carrier proteins and its derivatives and homologues thereof. Biotin binding protein other than streptavidin are well known in the art and the use of all such protein is contemplated herein.

Preferably, the solid matrix used in the methods of the invention permits the sequential application of reagents to a reaction molecule without complicated and time-consuming purification steps.

In this method, the opposite end of each nucleic acid segment is shared between each of the initial template precursors for a given nucleic acid segment to be detected or analyzed. Each initial template precursor is attached to a solid matrix. A wide range of methods have been used to bind DNA to a solid matrix. If the template precursor is a PCR product, one primer can contain a moiety that is used to attach the PCR product to a solid matrix. For example, this primer can contain a biotin moiety or another reactive moiety such as an amine group or thiol group, permitting the attachment of the PCR product to a solid matrix (Syvanen et al., 1988; Stamm and Brosius, 1991; Lund et al., 1988; Fahy et al., 1993; Kohsaka et al., 1993). The solid matrix can be either immobile or dispersible. For example, for a DNA segment with a biotinylated end, an immobile solid matrix can be an avidin or streptavidin coated microtiter plate (Jeltsch et al., 1993; Holmstrom et al, 1993) or manifold support (Lagerkvist et al., 1994). The most readily available dispersible solid matrix is beads that can be suspended through shaking. Beads can be designed to be magnetically pelleted (Lund et al., 1988; Hultman et al., 1989; Dawson et al., 1989) or they can be pelleted through centrifugation (Syvanen et al., 1988; Stamm and Brosius, 1991). Use of a dispersible solid matrix diminishes steric obstacles in enzymatic reactions, and facilitates removal of a small aliquot to be amplified. An alternative approach that allows a small aliquot of a reaction to be removed and used as a template for amplification is to use a method of reversible capture. Reversible capture can be accomplished by using a cleavable linkage arm (such as a chemically cleavable linkage arm or a photocleavable linkage arm (Dawson, 1989; Olejnik et al., 1996), by using a primer-encoded DNA binding domain that can be unbound by denaturation (Lew et al., 1989; Kemp et al., 1989; Kemp, 1992), or by the generation of a single stranded end during PCR, as such an end can reversibly anneal to its complement that is bound to a solid phase (Newton et al., 1993; Khudyakov et al., 1994).

In a particularly preferred embodiment, the kit further comprises an internal control in the form of either or both of an internal positive control and an internal negative control. Particularly useful controls are exons, introns and 3′ and 5′ untranslated regions of genes.

The present invention is further described by the following non-limiting Examples.

To demonstrate the utility of the technique, a number of mammalian cell lines were transfected with different plasmid constructs capable of expressing specific mRNAs. The cell lines were then cultured in selectable growth media until stable clones could be isolated. These transgenic cell lines were then grown and used to demonstrate the utility of the technique. Furthermore, in most cases, three transcripts were targeted: (i) the mRNA of a transgene, (ii) the mRNA of the endogenous gene from which the transgene was derived, and (iii) the mRNA of an endogenous ‘housekeeping’ gene. The housekeeping gene was detected as a duplex real-time PCR reaction in combination with the transgene and the endogenous gene, both as the product of a nuclear run-on and from total polyadenylated mRNA. Duplex reactions allow for quantitative, across sample comparisons.

### EXAMPLE 1

### Cell Lines, Transfection and Growth Conditions

Details of the plasmids referred to below are described in International Patent Application Nos. PCT/AU99/00195 and PCT/AU01/00297. Transgenic and parental cell lines were maintained in a range of tissue culture vessels, however, run-on were routinely performed from T75 vessels. The protocol was then optimized for six-well plates.

### EXAMPLE 2

### Porcine Kidney Cells—Type PK-1

Transformations were performed in 6 well tissue culture vessels (Nunc). Individual wells were seeded with 1×10⁵ PK-1 cells in 2 mL of Dulbecco's Modified Eagle Medium (DMEM) (GibcoBRL), 10% v/v fetal bovine serum (FBS) (GibcoBRL) and incubated at 37° C., 5% v/v CO₂ until the monolayer was 60-90% confluent, typically 16-24 hours.

To transform a single plate (6 wells), 12 μg of plasmid DNA (pCMV.EGFP) and 108 μL of GenePORTER 2 (trademark) (Gene Therapy Systems) were diluted into OPTI-MEM (trademark) (GibcoBRL) to obtain a final volume of 6 mL and incubated at room temperature for 45 minutes.

The tissue growth medium was removed from each well and the monolayer therein washed with 1 mL of 1×phosphate buffer saline (PBS) (Sigma) and the supernatant removed. The monolayer were overlaid with 1 mL of the plasmid DNA/GenePORTER conjugate for each well and incubated at 37° C., 5% v/v CO₂ for 4.5 hours.

1 mL of OPTI-MEM supplemented with 20% v/v FBS was added to each well and the vessel incubated for a further 24 hours, at which time the monolayer were washed with 1×PBS and media was replaced with 2 mL of fresh DMEM including 10% v/v FBS.

Forty-eight hours after transfection, the media was removed, the cell monolayer washed with 1×PBS as above, and 4 mL of fresh DMEM containing 10% v/v FBS, supplemented with 1.5 mg/mL Geneticin (registered trademark) (GibcoBRL) was added to each well. Geneticin was included in the media to select for stable transformed cell lines. The DMBM, 10% v/v FBS, 1.5 mg/l Geneticin media was changed every 48-72 hours. Cells transformed with pCMV.EGFP were examined after 24-48 hours for transient Enhanced Green Fluorescent Protein (EGFP) expression using fluorescence microscopy at a wavelength of 500-550 nm.

### EXAMPLE 3

### Madin Darby Kidney Cells—Type CRIB-1

Transformations were performed in six-well tissue culture vessels. Individual wells were seeded with 2×10⁵ CRIB-1 cells in 2 mL of DMEM, 10% v/v donor calf serum (DCS) (Gibco BRL) and incubated at 37° C., 5% v/v CO₂ until the monolayer was 60-90% confluent, typically 16-24 hours.

Prepare the following solutions in 10 mL sterile tubes:—

Solution A: For each transfection, dilute 1 μg of plasmid DNA (pCMV.BEV2.BGI2.2VEB, pCMV.BEV2.GFP.2VEB, pCMV.EGFP) into 100 μL of OPTI-MEM reduced serum medium (serum-free medium); and

Solution B: For each transfection, dilute 10 μl of LIPOFECTAMINE (trademark) (GibcoBRL) reagent into 100 μL OPTI-MEM I (trademark) reduced serum medium.

Combine the two solutions, mix gently and incubate at room temperature for 45 minutes to allow DNA-liposome complexes to form. While complexes form, rinse the CRIB-1 cells once with 2 mL of OPTI-MEM I reduced serum medium.

For each transfection, add 0.8 mL of OPTI-MEM I reduced serum medium to the tube containing the complexes. Mix gently and overlay the diluted complex solution onto the rinsed CRIB-1 cells. Incubate the cells with the complexes at 37° C. and 5% v/v CO₂ for 16-24 hours.

Remove transfection mixture and overlay the CRIB-1 monolayer with 2 mL of DMEM, 10% w/v DCS. Incubate the cells at 37° C. and 5% v/v CO₂ for approximately 48 hours. To select for stable transformants, replace the media every 72 hours with 4 mL of DMEM, 10% w/v DCS, 0.6 mg/mL Geneticin. Cells transformed with the transfection control pCMV.EGFP were examined after 24-48 hours for transient EGFP expression using fluorescence microscopy at a wavelength of 500-550 nm. After 21 days of selection, transgenically stable CRIB-1 colonies were apparent.

### EXAMPLE 4

### Murine Cells—Melanoma Type B16

Transformations were performed in six-well tissue culture vessels. Individual wells were seeded with 1×10⁵ cells in 2 mL of DMEM, 10% v/v FCS and incubated at 37° C., 5% v/v CO₂ until the monolayer was 60-90% confluent, typically 16-24 hours.

Prepare the following solutions in 10 mL sterile tubes:—

Solution A: For each transfection, dilute 1 μg of plasmid DNA (pCMV.EGFP, pCMV.TYR.BGI2.RYT) into 100 μL of OPTI-MEM I reduced serum medium; and

Solution B: For each transfection, dilute 10 μL of LIPOFECTAMINE reagent into 100 μL OPTI-MEM I reduced serum medium.

Combine the two solutions, mix gently and incubate at room temperature for 45 minutes to allow DNA-liposome complexes to form. While complexes form, rinse the cells once with 2 mL of OPTI-MEM I reduced serum medium.

For each transfection, add 0.8 mL of OPTI-MEM I reduced serum medium to the tube containing the complexes. Mix gently and overlay the diluted complex solution onto the rinsed cell monolayer. Incubate the cells with the complexes at 37° C. and 5% v/v CO₂ for 3-4 hours.

Remove transfection mixture and overlay the monolayer with 2 mL of DMEM, 10% w/v FCS. Incubate the cells at 37° C. and 5% v/v CO₂ for approximately 48 hours. To select for stable transformants, replace the media every 72 hours with 4 mL of DMEM, 10% w/v FCS, 1.0 mg/mL Geneticin. Cells transformed with the transfection control pCMV.EGFP were examined after 24-48 hours for transient EGFP expression using fluorescence microscopy at a wavelength of 500-550 nm. After 21 days of selection, transgenically stable NIH/3T3 or B16 colonies were apparent.

### EXAMPLE 5

### Human Cells—Melanoma Type MM96L and Breast Cancer Type MDA-MB-468

Transformations were performed in six-well tissue culture vessels. Individual wells were seeded with 1×10⁵ cells (MM96L) or 4×10⁵ cells (MDA-MB-468) in 2 mL of RPMI 1640 media (GibcoBRL), 10% v/v w/v FCS and incubated at 37° C., 5% v/v CO₂ until the monolayer was 60-90% confluent, typically 16-24 hours.

Prepare the following solutions in 10 mL sterile tubes:—

Solution A: For each transfection, dilute 1 μg of plasmid DNA (pCMV.EGFP, pCMV.BRN2.BGI2.2RNB, pCMV.HER2.BGI2.2REH) into 100 μL of OPTI-MEM I reduced serum medium; and

Solution B: For each transfection, dilute 10 μL of LIPOFECTAMINE reagent into 100 μL OPTI-MEM I reduced serum medium.

Combine the two solutions, mix gently and incubate at room temperature for 45 minutes to allow DNA-liposome complexes to form. While complexes form, rinse the cells once with 2 mL of OPTI-MEM I reduced serum medium.

For each transfection, add 0.8 mL of OPTI-MEM I reduced serum medium to the tube containing the complexes. Mix gently and overlay the diluted complex solution onto the rinsed cell monolayer. Incubate the cells with the complexes at 37° C. and 5% v/v CO₂ for 3-4 hours.

Remove transfection mixture and overlay the monolayer with 2 mL of RPMI 1640, 10% w/v FCS. Incubate the cells at 37° C. and 5% v/v CO₂ for approximately 48 hours. To select for stable transformants, replace the media every 72 hours with 4 mL of RPMI 1640, 10% w/v FCS and 0.6 mg/mL geneticin. Cells transformed with the transfection control pCMV.EGFP were examined after 24-48 hours for transient EGFP expression using fluorescence microscopy at a wavelength of 500-550 nm. After 21 days of selection, transgenically stable MM96L or MDA-MB-468 colonies were apparent.

The two methods outlined in Examples 6 and 7 represent examples of methods for the preparation of large number of nuclei.

### EXAMPLE 6

### Nuclei Preparation for Adherent Cell Types from a T75 Tissue Culture Vessel

Seed a T75 tissue culture vessel (Nunc) containing 30 mL of growth media (e.g. DMEM or RPMI 1640, including 10% v/v FBS) with 4×10⁶ cells and incubate at 37° C. and 5% v/v CO₂ until the monolayer is 90% confluent (overnight). Chill the monolayer by placing the T75 on a bed of ice. Decant medium and add 8 mL of ice-cold PBS to the T75 and wash the tissue monolayer by gently rocking the T75. Decant the PBS and repeat washing of the tissue monolayer with 1×PBS. Decant the PBS.

Overlay the tissue monolayer with 4 mL of ice-cold Sucrose Buffer 1 and incubate cells on ice for 2 minutes to lyse. Using a cell scraper, dislodge adherent cells. Examine a small aliquot of cells by phase-contrast microscopy. If the cells have not lysed, transfer them to an ice-cold dounce homogenizer (Braun). Break the cells with 5-10 strokes of a type S pestle. Additional strokes may be required. Examine microscopically to see if the nuclei are free from the cytoplasmic debris. Add 4 mL of ice-cold sucrose buffer 2 to the T75. Mix the buffers by gentle stirring with the cell scraper.

Add 4.4 mL ice-cold Sucrose Buffer 2 to a polyallomer SW41 tube (9/16×3¾ inch, Beckman) for SW41 rotor. Sucrose buffer 2 serves as the cushion. Unlysed cells will not sediment through the sucrose cushion. If these conditions do not result in a nuclear pellet, adjust the concentration of sucrose in sucrose buffer 2. Carefully layer the nuclei-containing sucrose buffer onto the sucrose cushion. Use ice-cold sucrose buffer 1 to top off the gradient. Do not centrifuge more than 2×10⁸ nuclei per tube.

Centrifuge the gradient for 45 minutes at 4° C. and 30,000×g (13300 rpm in SW41 rotor). Aspirate supernatant away from nuclei pellet. Return to ice bucket. Nuclei should form a tight pellet at the bottom of the tube and there may be some debris caught at the interface between sucrose buffers 1 and 2. If the cells did not lyse during dounce homogenization, nuclei will not pellet. Thus, it is important to be sure that the majority of the cells are clearly lysed. If the pellet appears as a gelatinous mass, nuclei have lysed and the pellet should be discarded.

Loosen nuclear pellet by gently vortexing for 5 seconds. Add 200 μL ice-cold glycerol storage buffer per 5×10⁷ nuclei and suspend nuclei by trituration. Nuclei will be clumped at first but will disperse with continued trituration. Trituration should be steady but should not create air bubbles. Aliquot 100 μL (approx 2.5×10⁷ nuclei) into chilled 2 mL microfuge tubes (Eppendorf). The addition of 40 units of an RNAse inhibitor (e.g. Rnasin, Promega) may be beneficial to protect the RNA. Immediately place in dry ice. Store frozen nuclei at −70° C. or in liquid nitrogen. Frozen nuclei are stable for at least 1 year.

### EXAMPLE 7

### Nuclei Preparation of Non-adherent Cell Types from a T75 Tissue Culture Vessel

Seed a T75 tissue culture vessel containing 30 mL of growth media (DMEM or RPMI 1640, including 10% v/v FBS) with 4×10⁶ cells and incubate at 37° C. and 5% v/v CO₂ overnight.

Transfer the contents of the T75 to a 50 mL screw-capped tube (Falcon) and place the tube on ice and allow to chill before processing. Centrifuge the tube for 5 minutes at 500×g and 4° C. to pellet cells. Decant medium and add 10 mL of ice-cold 1×PBS to the tube and suspend the cells by gentle trituration. Decant the PBS and repeat washing of the cells with 1×PBS. Decant the PBS.

Suspend the cells in 4 mL of ice-cold sucrose buffer 1 and incubate cells on ice for 2 minutes to lyse. Examine a small aliquot of cells by phase-contrast microscopy. If the cells have not lysed, transfer them to an ice-cold dounce homogenizer. Break the cells with 5-10 strokes of a type S pestle and return the cells to the tube. Additional strokes may be required. Examine microscopically to see if the nuclei are free from the cytoplasmic debris.

Add 4 mL of ice-cold sucrose buffer 2 to the tube. Mix the buffers by gentle trituration. The final concentration of sucrose in cell homogenate should be sufficient to prevent a large build up of debris at the interface between homogenate and the sucrose cushion. The amount of sucrose buffer 2 added to cell homogenate may need to be adjusted.

Add 4.4 mL ice-cold sucrose buffer 2 to a polyallomer SW41 tube (9/16×3¾ inch, Beckman) for SW41 rotor. Sucrose buffer 2 serves as the cushion. Unlysed cells will not sediment through the sucrose cushion. If these conditions do not result in a nuclear pellet, adjust the concentration of sucrose in sucrose buffer 2.

Carefully layer the nuclei-containing sucrose buffer nuclei onto the sucrose cushion. Use ice-cold sucrose buffer 1 to top off the gradient. Do not centrifuge more than 2×10⁸ nuclei per tube. Centrifuge the gradient 45 minutes at 30000×g (13300 rpm in SW41 rotor), 4° C. Aspirate supernatant away from nuclei pellet. Return to ice bucket. Nuclei should form a tight pellet at the bottom of the tube and there may be some debris caught at the interface between sucrose buffers 1 and 2. If the cells did not lyse during dounce homogenization, nuclei will not pellet. Thus, it is important to be sure that the majority of the cells are clearly lysed. If the pellet appears as a gelatinous mass, nuclei have lysed and the pellet should be discarded.

Loosen nuclear pellet by gently vortexing 5 seconds. Add 100 μL ice-cold Glycerol Storage Buffer per 5×10⁷ nuclei and suspend nuclei by trituration. Nuclei will be clumped at first but will disperse with continued trituration. Trituration should be steady but should not create air bubbles.

Aliquot 100 μL (approx 1-2.5×10⁷ nuclei) into chilled 2 mL microfuge tube. The addition of 40 units of an RNAse inhibitor may be beneficial to protect the RNA. Immediately place in dry ice. Store frozen nuclei at −70° C. or in liquid nitrogen. Frozen nuclei are stable for at least 1 year.

### EXAMPLE 8

### Buffers

Glycerol Storage Buffer:

40% v/v glycerol (Univar)

4% RNAsecure (Ambion) (optional)

50 mM Tris-Cl pH 8.3 (ICN Biomedicals, Inc)

5 mM magnesium chloride (BDH)

0.1 mM EDTA (Univar)

4 mM phenylmethylsulfonyl fluoride (PMSF, Sigma) from 0.1 M stock in isopropanol

Sucrose Buffer 1:

The molarity of sucrose required to differentially sediment nuclei is determined empirically for each cell type. The molarity should be sufficient to cause cell debris to remain in suspension whilst nuclei sediment. A layer of cell debris at the buffer-interface will interfere with the proper sedimentation of the nuclei. 0.32 M sucrose works well for most cell types.

0.32 M sucrose (Sigma)

0.1 mM EDTA

0.5% Igaepal CA630 (Sigma)

1.0 mM 1,4-Dithiothreitol (DTT, Roche)

10 mM Tris-Cl pH 8.0

0.1 mM PMSF from 0.1 M stock in isopropanol

1 mM N,N,N′N′-tetraacetic acid (EGTA, Sigma)

1 mM Spermidine (Sigma)

Add Igaepal and DTT to buffer just before use from 1 M stock solution.

Sucrose Buffer 2:

The molarity of sucrose required to differentially sediment nuclei is determined empirically for each cell type. 1.7 M sucrose works well for most cell types, however, typically the correct molarity occurs in the range of 1.5-2.2 M sucrose.

1.7 M sucrose

5.0 mM magnesium acetate (Sigma)

0.1 mM EDTA

1 mM DTT

10 mM Tris-CL pH 8.0

0.1 mM PMSF from 0.1 M stock in isopropanol

Add DTT to buffer just before use from 1 stock solution.

### EXAMPLE 9

Kit

This kit comprises the necessary components for the preparation of a suitable eukaryotic, prokaryotic or virus RNA template from mammalian cells for quantitative real-time PCR. The kit includes a method for isolation of nuclei from cells grown in a six-well tissue culture plate as either adherent or non-adherent cells. Typically, a confluent well contains approximately 10⁶ cells.

### Empirical Determination of the Cell Lyses and Nuclei Wash Buffers for Adherent Cell Types

The kit includes two sets of solutions (solution A and B; solution C and D), that when combined, form either the cell lyses buffer or nuclei wash buffer respectively. This ratio needs to be empirically determined only for solutions A and B. The ratio of solutions C and D is the same as that used for solutions A and B.

Seed 2×6 well tissue culture plates containing 2 mL of growth media (e.g. DMEM or RPMI 1640, including 10% v/v FBS) with 4×10⁵ cells and incubate at 37° C. and 5% v/v CO₂ until the monolayer is 90% confluent (overnight). The growth media and cell-seeding rate will depend on the cell type grown. Preferably, 10⁶ cells should be available for processing of each sample.

The following day, prepare aliquots of the cell lyses buffer as described below.

Number 11 microfuge tubes and aliquot into each tube the amount of solution A and B as set out in the table below.

| Tube Number | Solution A | | Solution B | |
|---|---|---|---|---|
| 1 | 0 | μL | 1000 | μL |
| 2 | 100 | μL | 900 | μL |
| 3 | 200 | μL | 800 | μL |
| 4 | 300 | μL | 700 | μL |
| 5 | 400 | μL | 600 | μL |
| 6 | 500 | μL | 500 | μL |
| 7 | 600 | μL | 400 | μL |
| 8 | 700 | μL | 300 | μL |
| 9 | 800 | μL | 200 | μL |
| 10 | 900 | μL | 100 | μL |
| 11 | 1000 | μL | 0 | μL |

Chill the monolayer of cells by placing the plates on a bed of ice. Aspirate away the medium and add 2 mL of ice-cold 1×PBS to the to each well and wash the tissue monolayer by gently rocking the plate. Aspirate away the PBS and repeat washing of the tissue monolayer with 1×PBS. Aspirate away the PBS.

Overlay the tissue monolayer in each of 11 wells with 1 mL of the prepared of ice-cold cell lysis buffers (microfuge tubes 1 to 11). Overlay the twelth well with PBS. The cells in the twelfth well may be used as a representative sample of unlysed cells for comparison to with lysed cells. Incubate cells on ice for 2 minutes to lyse. Use a cell scraper to dislodge and assist with cell lyses. Examine a small aliquot of cells by phase-contrast microscopy. If the cells have not lysed, transfer them to an ice-cold dounce homogenizer (Braun). Break the cells with 5-10 strokes of a type S pestle. Additional strokes may be required. Examine microscopically to see if the nuclei are free from the cytoplasmic debris. Transfer the cell lysate to an ice-cold 2 mL microfuge tube and centrifuge for 15 minutes at 4° C. and 2,500×g. The lysis buffers (numbered 1 to 11) increases in density. A nuclei/cell debris pellet will be apparent in tube number 1 and should be absent in tube number 11. The tube containing the largest pellet of relatively clean, intact, nuclei represent the most suitable ratio of solutions (A and B; C and D) for the specific cell type evaluated.

Solution A

1.7 M sucrose

5.0 mM magnesium acetate

0.1 mM EDTA

0.5% Igaepal CA630

1 mM DTT

10 mM Tris-CL pH 8.0

0.1 mM PMSF from 0.1 M stock in isopropanol

Solution B

0.32 M sucrose

0.1 mM EDTA

0.5% Igaepal CA630

1.0 mM DTT

10 mM Tris-Cl pH 8.0

0.1 mM PMSF from 0.1 M stock in isopropanol

1 mM EGTA

1 mM Spermidine

Solution C

1.7 M sucrose

5.0 mM magnesium acetate

0.1 mM EDTA

1 mM DTT

10 mM Tris-CL pH 8.0

0.1 mM PMSF from 0.1 M stock in isopropanol

Solution D

0.32 M sucrose

0.1 mM EDTA

1.0 mM DTT

10 mM Tris-Cl pH 8.0

0.1 mM PMSF from 0.1 M stock in isopropanol

1 mM EGTA

1 mM Spermidine

### Empirical Determination of the Cell Lyses and Nuclei Wash Buffers for Non-Adherent Cell Types

The kit includes two sets of solutions (solution A and B; solution C and D), that when combined, form either the cell lyses buffer or nuclei wash buffer respectively. This ratio needs to be empirically determined only for solutions A and B to make the for the cell lysis buffer. The ratio of solutions C and D is the same as that used for solutions A and B.

Seed 2×6 well tissue culture plates containing 2 mL of growth media (e.g. DMEM or RPMI 1640, including 10% v/v FBS with 4×10⁵ cells and incubate at 37° C. and 5% v/v CO₂ overnight. The growth media and cell-seeding rate will depend on the cell type grown. Preferably, 10⁶ cells are available for processing for each sample.

The following day, prepare aliquots of the cell lyses buffer as described in Example 9.

Number 12 2 mL microfuge tubes 1 to 12 and transfer the contents of each well to one of the 12 numbered microfuge tube and place the tubes on ice and allow to chill before processing. Centrifuge the tubes for 5 minutes at 4° C. and 500×g to pellet cells. Aspirate away the medium and add 1.5 mL of ice-cold 1×PBS to the tube and suspend the cells by gentle trituration. Centrifuge the tubes for 5 minutes at 4° C. and 500×g to pellet cells. Aspirate away the PBS and repeat washing of the cells with 1×PBS. Aspirate away the PBS.

Overlay the cell pellets in tubes 1 to 11 with 1 mL of the appropriate ice-cold cell lysis buffers. Overlay the pellet in the twelfth tube with PBS. The cells in the twelfth tube may be used as a representative sample of unlysed cells for comparison to with lysed cells. Incubate cells on ice for 2 minutes to lyse. Gently triturate the cells to resuspend and assist with cell lyses. Examine a small aliquot of cells by phase-contrast microscopy. If the cells have not lysed, transfer them to an ice-cold dounce homogenizer (Braun). Break the cells with 5-10 strokes of a type S pestle. Additional strokes may be required. Return the cell lysate to their respective tube and centrifuge the tubes for 15 minutes at 4° C. and 2,500×g. The cell lysis buffers (numbered 1 to 11) increases in density. A nuclei/cell debris pellet will be apparent in tube number 1 and should be absent in tube number 11. The tube that yields the greatest number of relatively clean, intact nuclei represents the ratio of Buffers A and B, and Buffers C and D that is most suitable for the specific cell type evaluated.

### EXAMPLE 10

### Nuclei Preparation of Adherent Cell Types from a Six-well Tissue Culture

Seed each well of a six-well tissue culture plate containing 2 mL of growth media (e.g. DMEM or RPMI 1640, including 10% v/v FBS with 4×10⁵ cells and incubate at 37° C. and 5% v/v CO₂ until the monolayer is 90% confluent (overnight). The growth media and cell-seeding rate will depend on the cell type grown. Preferably, 10⁶ cells are available for processing for each sample.

Chill the tissue monolayer by placing the plate on a bed of ice. Aspirate away the medium and add 2 mL of ice-cold 1×PBS to each well and wash the tissue monolayer by gently rocking the plate. Aspirate away the PBS and repeat washing of the tissue monolayer with 1×PBS. Aspirate away the PBS.

Overlay the tissue monolayer with 1 mL of the appropriate ice-cold cell lysis buffer as determined above and incubate cells on ice for 2 minutes to lyse. Use a cell scraper to dislodge and assist with cell lyses. Examine a small aliquot of cells by phase-contrast microscopy. If the cells have not lysed, transfer them to an ice-cold dounce homogenizer (Braun). Break the cells with 5-10 strokes of a type S pestle. Additional strokes may be required. Examine microscopically to see if the nuclei are free from the cytoplasmic debris. Transfer the cell lysate to a cold 2 mL centrifuge tube and centrifuge for 15 minutes at 4° C. and 2500×g. Remove the supernatant. Add 1 mL of the appropiate ice-cold wash buffer as determined above and gently resuspend the nuclei. Centrifuge the nuclei suspension for 15 minutes at 4° C. and 2500×g.

Aspirate supernatant away from nuclei pellet. Add 100 μL ice-cold glycerol storage buffer and suspend nuclei by gentle trituration. Nuclei will be clumped at first but will disperse with continued trituration. Trituration should be steady but should not create air bubbles. The addition of 40 units of an RNAse inhibitor may be beneficial to protect the RNA. Immediately place in dry ice. Store frozen nuclei at −70° C. or in liquid nitrogen. Frozen nuclei are stable for at least 1 year.

### EXAMPLE 11

### Nuclei Preparation of Non-adherent Cell Types from a Six-well Tissue Culture Plate

Seed each well of a six-well tissue culture plate (Nunc) containing 2 mL of growth media (e.g. DMEM or RPMI 1640, including 10% v/v FBS) with 4×10⁵ cells and incubate overnight at 37° C. and 5% v/v CO₂. Preferably, 10⁶ cells are available for processing for each sample.

Transfer the contents of each well to a 2 mL microfuge tube and place the tube on ice to chill before processing. Centrifuge the tube for 5 minutes at 4° C. and 500×g to pellet cells. Aspirate away the medium and add 1.5 mL of ice-cold 1×PBS to the tube and suspend the cells by gentle trituration. Centrifuge the tube for 5 minutes at 4° C. and 500×g to pellet cells. Aspirate away the PBS and repeat washing of the cells with 1×PBS. Aspirate away the PBS.

Suspend the cells in 1 mL of the appropriate ice-cold lyses buffer as determined above and incubate cells on ice for 2 minutes to lyse. Gently triturate the cell lysate to assist in disruption of the cells. Examine a small aliquot of cells by phase-contrast microscopy. If the cells have not lysed, transfer them to an ice-cold dounce homogenizer. Break the cells with 5-10 strokes of a type S pestle and return the cells to the tube. Additional strokes may be required. Examine microscopically to see if the nuclei are free from the cytoplasmic debris.

Centrifuge the cell lysate for 15 minutes at 4° C. and 2500×g. Remove the supernatant. Add 1 mL of the appropriate ice-cold sucrose wash buffer as determined above and gently suspend the nuclei. Centrifuge the suspended nuclei for 15 minutes at 4° C. and 2,500×g. Aspirate away the supernatant.

Loosen nuclear pellet by gently vortexing 5 seconds. Add 100 μL ice-cold glycerol storage buffer and suspend the nuclei by gentle trituration. Nuclei will be clumped at first but will disperse with continued trituration. Trituration should be steady but should not create air bubbles.

The addition of 40 units of an RNAse inhibitor may be beneficial to protect the RNA. Immediately place in dry ice. Store frozen nuclei at −70° C. or in liquid nitrogen. Frozen nuclei are stable for at least 1 year.

### EXAMPLE 12

### Standard Biotin-16-UTP Run-on Reaction

Add to 100 μL of nuclei (10⁷ for T75 vessel or 10⁶ for 6-well plate) in ice cold, glycerol storage buffer, 100 μL of reaction buffer containing ribonucleoside triphosphates. Incubate for 20 minutes at 30° C. with gentle shaking or slow rotation (6 rpm).

Lyse the nuclei and initiate DNA digestion by adding 20 μL 20 mM calcium chloride (Sigma) and 10 μL of 10 mg/mL RNAse-free DNAse 1 (Roche). Incubate for 30 minutes at 37° C. with gentle shaking or slow rotation.

Add 25 μL of 10×SET and 5 μL of 10 mg/mL transfere RNA (tRNA, Roche). Initiate peptide hydrolysis by adding 2 μL of 10 mg/mL proteinase K (Roche). Incubate the samples at 37° C. for 30 minutes with gentle shaking or slow rotation.

Add 1 mL of Trizol (Life Technologies) reagent, shake vigorously by hand for 15 seconds and incubate at 30° C. for 3-5 minutes to denature proteins with gentle shaking or slow rotation.

Add 200 μL of chloroform, shake vigorously for 15 seconds and incubate at 30° C. for 3-5 minutes with gentle shaking or slow rotation.

Centrifuge the samples for 15 minutes at 2-8° C. and 12000×g. Transfer the aqueous phase to a fresh microfuge tube. Avoid the interface between the aqueous and phenol phases.

Add 1 mL of isopropanol and mix by inverting the sample. Incubate the sample at 15-30° C. for 10 minutes and then centrifuge for 10 minutes at 2-8° C. and 12000×g. Remove supernatant from RNA pellet and add 1 mL of RNAse-free 75% v/v ethanol (BDH) (diluted with Diethyl Pyrocarbonate (DEPC-treated) (Sigma) H₂O). Vortex briefly to resuspend pellet. The pellet should break apart but still remain as small pieces.

Centrifuge for 5 minutes at 7500×g to pellet RNA. Remove the supernatant. Air-dry the pellet to remove ethanol, but do not over dry. Add 20 μL of RNAse-free H₂O (DEPC-treated) and dissolve the RNA pellet. Store the RNA pellet at −70° until further processing.

2×Nuclear Run-on Reaction Buffer

100 mM Tris-Cl pH 8.0

50 mM KCl (Sigma)

600 mM (NH₄)₂SO₄ (Sigma)

2 mM MgCl₂

2 mM MnCl₂.4H₂O (Sigma)

2mM DTT

10 mM Spermidine

0.2% N-lauroylsarcosine (Sigma)

10% v/v glycerol

4% RNAsecure

1 mM ATP (Roche)

1 mM GTP (Roche)

1 mM CTP (Roche)

150 uM UTP (Roche)

40 uM biotin-16-UTP (Roche)

Make up 2×reaction buffer without the rNTPs including the 4% RNAsecure and incubate at 60° C. for 10 minutes to inactivate RNAse A. Then cool on ice and add rNTPs.

10×set

5% w/v sodium dodecyl sulfate (Sigma)

50 mM EDTA

100 mM Tris-Hcl, pH 7.4

### EXAMPLE 13

### Purification of Biotin Labeled RNA Using the Dynal Dynabeads KilobasesBINDER (trademark) Kit

Purification of the biotin labeled RNA uses the standard protocol for the purification of biotin labeled nucleic acids as described in the protocol of the Dynabeads kilobaseBINDER (trademark) Kit (Dynal Product Number 601.01).

Resuspend the Dynabeads M-280 streptavidin by shaking the vial to obtain a homogeneous suspension.

Transfer 10 μL (100 μg) per sample of the resuspended Dynabeads to an 1.5 mL microfuge tube. Place the tube in a Dynal Magnetic Particle Concentrator (MPC) for 1-2 minutes or until the Dynabeads have settled on the tube wall.

Carefully remove the supernatant while the tube remains in the Dynal MPC. Avoid touching the Dynabead pellet.

Remove the tube from the Dynal MPC. Add twice the volume of wash solution A along the inside of the tube where the Dynabeads are collected and gently resuspending by pipetting (avoid foaming). Incubate at room temperature for 2-5 minutes. Place the tube in the Dynal MPC and remove the supernatant. Wash the Dynabeads once more with wash solution A and remove the supernatant. Remove the tube from the Dynal MPC.

Wash the beads in an equal volume of wash solution B twice as described above. Remove the tube from the Dynal MPC.

Resuspend the Dynabeads in 20 μL per sample of Binding Solution along the inside of the tube where the Dynabeads are collected and gently resuspending by pipetting (avoid foaming).

Place the tube in the Dynal MPC and remove the binding solution without touching the pellet. Remove the tube from the Dynal MPC.

Resuspend the Dynabeads in 20 μL binding solution per sample.

Add 20 μL of biotinylated RNA-fragments to 20 μL Dynabeads in binding solution. Mix carefully while avoiding foaming of the solution.

Incubate the samples at room temperature (15-25° C.) for 3 hours on a roller to keep the Dynabeads in suspension.

Carefully remove the supernatant while the tube remains in the Dynal MPC. Avoid touching the Dynabead pellet.

Wash the Dynabeads/RNA-complex twice in 40 μL washing solution C and once in RNAse-free H₂O or RNAse-free 10 mM Tris pH 8.0. Optional further washing may also occur.

Resuspend the Dynabeads/RNA-complex in 5 μL of RNAse-free H₂O or RNAse-free 10 mM Tris pH 8.0 per million cells harvested, for example, 10⁶ cell use 5 μL and 10⁷ use 50 μL.

Wash Solution A

DEPC-treated 0.1 M NaOH (Sigma)

DEPC-treated 0.05 M NaCl (Sigma)

Wash Solution B

DEPC-treated 0.1 M NaCl

Wash Solution C

RNAse-free 10 mM Tris-HCl (pH 7.5)

DEPC-treated 1 mM EDTA

DEPC-treated 2.0 M NaCl

### EXAMPLE 14

### Preparation of Poly A RNA for the Establishment of RNA Standard Curves

dT SS RNA (oligo dT purified steady state RNA/i.e. poly A RNA) was purified from a transgenic representative of the cell lines of interest and used for the establishment of standard curves and assay optimization.

Poly A mRNA was purified from 10 μg of total SS RNA using the Dynal Dynabeads mRNA Direct (trademark) Micro Kit (Prod #610.21) and then eluted from the beads in a predetermined volume.

For the purpose of establishing standard curves, mRNA quantities were expressed as total RNA equivalents.

### EXAMPLE 15

### Preparation of DNA for the Establishment of DNA Standard Curves

Genomic DNA was purified from a transgenic representative of the cell lines of interest and used for the establishment of DNA standard curves and assay optimization. Genomic DNA was purified using a Qiagen Genomic-tip 100/G (CAT #10243) as per manufacturer's protocol.

### EXAMPLE 16

### Quantitative Analysis of Nascent RNA Transcription Levels by Real-time PCR—Target Choice

Purified biotin-labeled nascent RNA transcripts (Example 13) were quantitatively measured by real-time PCR. The AB Applied Biosystems TaqMan PCR reporter chemistry and the Corbett Research Rotorgene 2000 real-time PCR Thermocycler and analysis instrument were employed.

Primers and probes were designed to target coding sequence within a single contiguous exon. To exlimplify the protocol, a number of gene transcript targets were chosen across a range of species. Glyceralderhyde phosphate dehydrogenase (GAPDH) and glucose-6-phosphate dehydrogenase (G6PD) were chosen as interal duplexing controls to verify the real-time PCR and to allow for across sample comparisons of transcription rates. Duplex is a real-time PCR technique wherein two different target molecules are amplified in the same reaction tube, the internal control (GAPDH or G6PD) and the specific endogenous or transgene target. Candiate cell lines were chosen for procine (Example 2), bovine (Example 3), murine (Example 4), and human (Example 5). The sequences of the primers and probes used in this Example are shown in Table 1.

TABLE 1

| | |
|---|---|
| Primers | PROBES |

| | | | |
|---|---|---|---|
| Name | Sequence 5′-3′ | Name | Sequence 5′-3′ |

| | | | |
|---|---|---|---|
| GAPDH Univ Fwd | CAAGGCTGTGGGCAAGGT | GAPDH Univ VIC | ATCCCTGAGCTGAACGG |
| | [SEQ ID NO:1] | (homosapien, | [SEQ ID NO:8] |
| | | porcine, bovine) | |
| GAPDH Univ Rev | GGAAGGCCATGCCAGTGA | | |
| (homosapien, murine, | [SEQ ID NO:2] | | |
| porcine, bovine) | | | |
| GAPDH Univ Fwd #4 | CAAGGCTGTGGGCAAGG | GAPDH murine VIC | ATCCCAGAGCTGAACGG |
| | [SEQ ID NO:3] | (murine) | [SEQ ID NO:9] |
| GAPDE Univ Rev #5 | CGGAAGGCCATGCCAGTGA | | |
| (homosapien, murine, | [SEQ ID NO:4] | | |
| porcine, bovine) | | | |
| G6PD-1 Fwd | GCCTTCTGCCCGAAAACAC | G6PD-1 VIC | TGGGCTATCCCCGTTCCCGC |
| (homosapien) | [SEQ ID NO:5] | (human) | [SEQ ID NO:10] |
| G6PD-1 Rev | TGCGGATGTCAGCCACTGT | | |
| (homosapien/bovine) | [SEQ ID NO:6] | | |
| G6PD-1 Fwd | GCCTTTTGCCCGAAGACAC | G6PD-1 VIC | TGGGCTATGCCCGCTCCCGC |
| (Bovine/Porcine) | [SEQ ID NO:7] | | [SEQ ID NO:11] |

To exemplify the protocol with regards to gene transcripts from exogenous transgenes, cells were transfected with plasmids containing the exogenous transgene placed operably under the human cytomegalovirus (CMV) immediate early promoter and terminated by the SV40 early mRNA polyadenylation signal. Stable expression clones were grown and these processed (Example 6, Example 12, Example 13) to produce biotin-labeled RNA template and their respective transcription levels quantified. Three plasmid constructs were used:—

(1) an inverted repeat of the bovine entrovirus RNA polymerase gene (BEV) interrupted by the human β-globin gene intron 2 (BGI2) placed between CMV and SV40 to produce the plasmid pCMV.BEV.BGI.VEB;

(2) the sequence of the enhanced green fluorescent protein (EGFP) placed between CMV and SV40 to produce the plasmid PCMV.EGFP; and

(3) an inverted repeat of a sub-region from the human HER2 gene interrupted by the human β-globin gene intron 2 (BGI2) placed between CMV and SV40 to produce the plasmid pCMV.HER2.BGI.2.REH.

The sequences of the primers and probes used are shown in Table 2.

TABLE 2

| | |
|---|---|
| Primers | PROBES |

| Name | Sequence 5′-3′ | Name | Sequence 5′-3′ |
|---|---|---|---|
| Exo SV40 Univ Fwd | GCCGCGACTCTAGATCATAATCA | SV40 Univ | AAACCTCTACAAATGTGGTA |
| | [SEQ ID NO:12] | Rev FAN | [SEQ ID NO:17] |
| Exo SV40 Univ Rev | TGTGGGAGGTTTTTTAAAGCAAGT | | |
| | [SEQ ID NO:13] | | |
| BEV Exo Fwd | GTACTCGATTTGTCCTGCCATTG | | |
| | [SEQ ID NO:14] | | |
| EGFP Exo Fwd | GGCATGGACGAGCTGTACAAG | | |
| (171 bp) | [SEQ ID NO:15] | | |
| HER2 Exo Fwd | GTAGAGGTGGCGGAGCATGT | | |
| | [SEQ ID NO:16] | | |

To exemplify the protocol with regards to gene transcripts from endogenous genes, stable expression clones were grown and these processed (Example 6, Example 12, Example 13) to produce biotin-labeled RNA template and their respective transcription levels quantified. The endogenous targets that were chosen were the human BRN2 and HER2 genes and the murine tyrosinase (TYR) gene.

The sequences of the primers and probes used are shown in Table 3.

TABLE 3

| | |
|---|---|
| Primers | PROBES |

| Name | Sequence 5′-3′ | Name | Sequence 5′-3′ |
|---|---|---|---|
| BRM-2 Endo 3′ | GGCTCTGGGCACCCTGTAT | BRN-2 3′ | CAACGTGTTCTCGCAGACCACCATCT |
| Fwd (5′mRNA) | [SEQ ID NO:18] | Endo 6FAM | [SEQ ID NO:24] |
| BRN-2 Endo 3′ | CAGCTGCAGGGCCTCAAA | | |
| Rev (5′mRNA) | [SEQ ID NO:19] | | |
| TYR Endo 3″Fwd | AACTGTGACATTTGCACAGATGAGT | TYR3′ Endo | TTGGGAGGTCGTCACCCTGAAAATCC |
| (5′mRNA) | [SEQ ID NO:20] | 6FAM | [SEQ ID NO:25] |
| TYR 3′ Endo Rev | GAAGGATGCTGGGCTGAGTAAGT | | |
| (5′MrNA) | [SEQ ID NO:21] | | |
| HER-2 Endo 5′ | GGACCTAGTCTCTGCCTTCTACTCTCTA | HER-2 Endo | CTGGCCCCCCTCAGCCCTACAA |
| Fwd | [SEQ ID NO:22] | 5′ FAN | [SEQ ID NO:26] |
| HER-2 Endo 5′ | GCCCCTCCCCACACTGA | | |
| Rev | [SEQ ID NO:23] | | |

### EXAMPLE 17

### Quantitative Analysis of Nascent RNA Transcription Levels by Real-time PCR

Reverse Transcription of RNA, Step One of 2-step RT-PCR

2.5-5 uL of Dynabead suspension of biotin-labeled and captured nascent RNA template was added to a RT reaction containing the following: RT buffer mix as described in Table 4, 80 nM of each gene specific reverse primer, AB (Applied Biosystems), multiscribe reverse transcriptase 0.5 U/ul (Cat #4311235), AB RNase inhibitor 0.4 U/ul (Cat #N808-0119), and to a final volume of 15 uL in a 0.1 mL Corbett Research Rotorgene PCR tube.

The samples were incubated as shown in Table 5.

TABLE 4

| 5x RT buffer mix | | | | RT Final concentration |
|---|---|---|---|---|
| Components | | Catalogue # | Vol Per Reaction | 15. μl |
| 10x Concentration | AB Gold PCR Buffer | AB#4306894 | 1.0 μL | 0.67x concentration |
| 25 mM | MgCl2 | AB #N808-0010 | 1.05 μL | 1.75 mM |
| 100 mM | dATP's | Roche # 1969064 | 0.03 μL | 0.2 mM |
| 100 mM | dCTP's | | 0.03 μL | 0.2 mM |
| 100 mM | dGTP's | | 0.03 μL | 0.2 mM |
| 100 mM | dTTP's | | 0.03 μL | 0.2 mM |
| 1,000 mM | DTT | | 0.15 μL | 10.0 mM |
| | StH2O/Balance | | 0.68 μL | |

| | |
|---|---|
| TOTAL RT Buffer Mix | 3.0 μL |

TABLE 5

| Reverse Transcription Step | | Temp | Time | #Cycles | Method |
|---|---|---|---|---|---|
| Cycle 1 | Step 1 | 25° C. | 10 min | 1 cycle | Manual |
| | Step 2 | 46° C. | 20 min | | heat blocks |
| | Step 3 | 94° C. | 5 min | | |
| | Step 4 | 25° C. | 5 min | | |

Quantitative PCR, Step 2 of RT-PCR

35 μL of PCR Mastermix was then pipetted into the RT Rxn ensuring the Dynabeads are thoroughly mixed. To give a final reaction volume of 50 μL with a 1×concentration of AB 2×Universal PCR Master Mix Cat #4304437 and TaqMan probes and primers as required for each example.

Amplification and quantitative detection was performed on a Corbett Research Rotorgene real-time PCR Thermocycler with the amplification protocol shown in Table 6.

TABLE 6

| PCR Step | | Temperature | Time | |
|---|---|---|---|---|
| Cycle 1 | Step 1 | 50° C. | 2 min | 1 Cycle |
| Cycle 2 | Step 1 | 95° C. | 10 min | 1 Cycle |
| Cycle 3 | Step 1 | 95° C. | 15 sec | 55 Cycles |
| | Step 2 | 60° C. | 45 sec | |

### EXAMPLE 18

### Modified Real-time PCR Protocol

The process is modified to enable the streptavidin Dynabead captured biotin UTP labeled transcripts to be cleaved or eluted off the Dynabead by the incorporation of a cleavable linker between either (a) the UTP and the biotin label; or (b) the Dynabead and the streptavidin.

The cleavable linker is a disulfide S-S bridge that could be disrupted by DTT (dithiothreiol; Cleland's reagent) which is a reducing reagent. DTT is compatible with TaqMan chemistry up to 10 mM.

The release of the biotin UTP labeled transcripts from the streptavidin Dynabead helps ensure a more homogenous sample and reaction as the dense Dynabeads sink rapidly and form a pellet during all steps of the process. This trait has the potential to cause inaccurate aliquoting of the sample and poor access of RT and PCR reagents to the nascent RNA transcripts and cDNA transcripts.

### EXAMPLE 19

### Results of Quantitation Real-time RT-PCR

Analyses were performed using the Rotogene real-time analysis software and the following genes have been detected from biotin-labeled nuclear-run (NRO) template (Table 7).

TABLE 7

| | | | Target Gene | Duplexed with Internal |
|---|---|---|---|---|
| | Species | Cell line | Of Interest | control Target Gene |
| Endogenous | | | | |
| Targets | | | | |
| | Human | MM96L | G6PD | Not Duplexed |
| | Human | MM96L | BRN 2 | GAPDH |
| | Mouse | B16 | Tyrosinase | GAPDH |
| | Human | MDA-MB | HER 2 | GAPDH |
| | | 468 | | |
| Transgene | | | | |
| Targets | | | | |
| | Mouse | B16 | EGFP Exo | GAPDH |
| | Human | MM96L | EGFP Exo | GAPDH |
| | Bovine | CRIB | BEV Exo | G6PD |

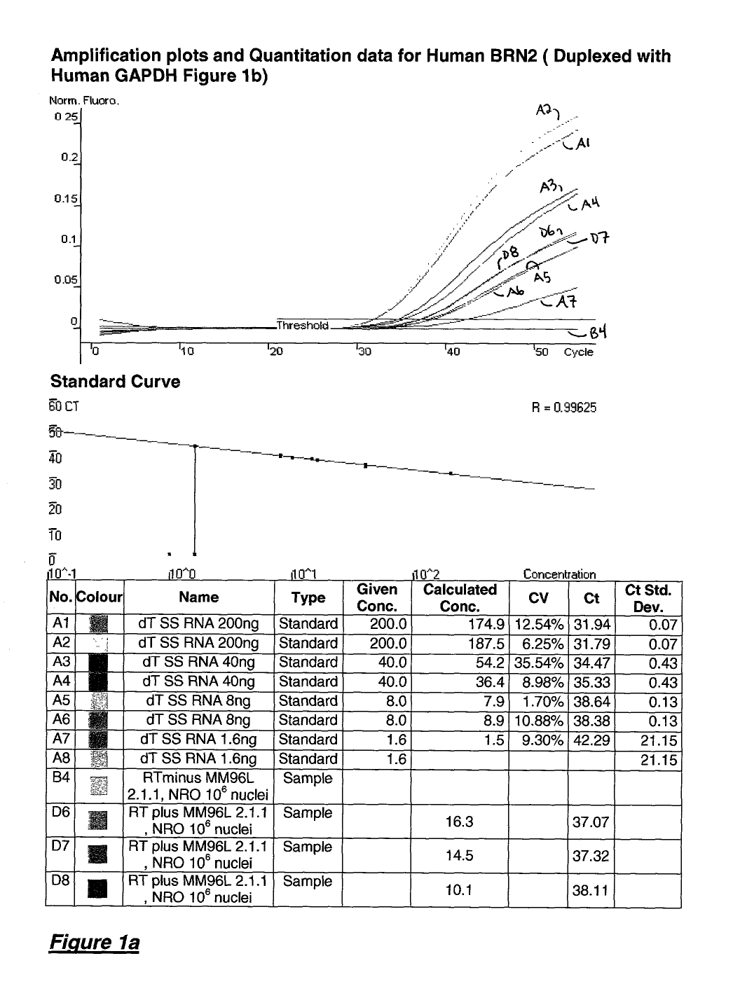

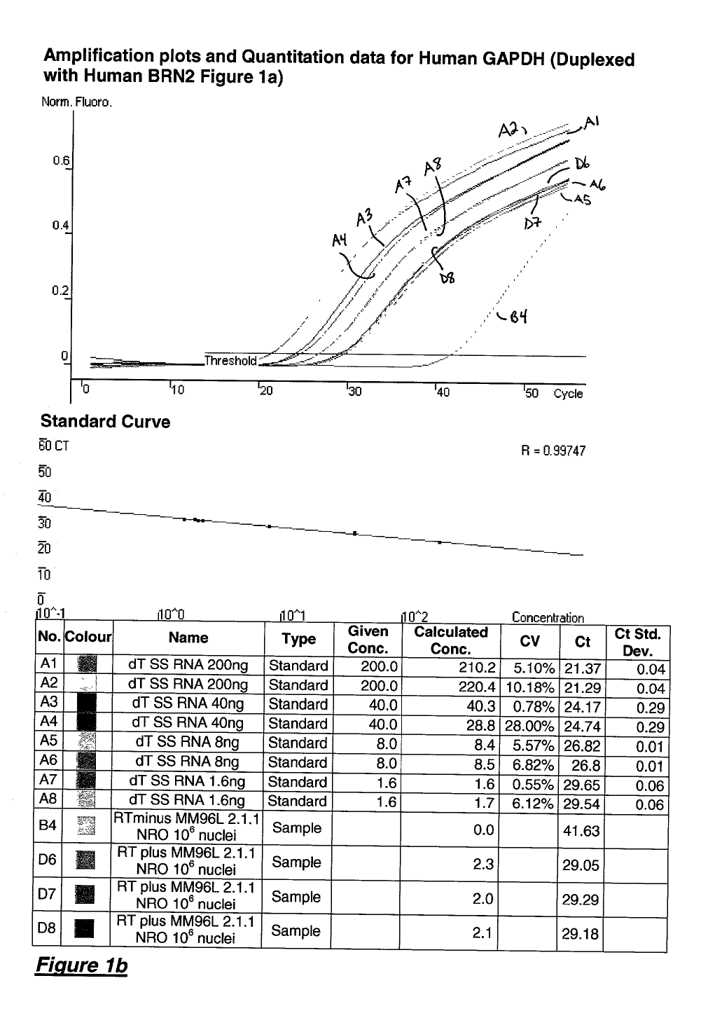

The results are shown in FIGS. 1a and 1b to FIGS. 6a and 6b.

In FIGS. 1a and 1b, the data exemplify the establishment of a mRNA standard curve's (samples A1 to A8) for the parental non-transgenic human cell line MM96L. To exemplify the quantitation of the template derived from a single NRO procedure, RT positive reactions (samples D6-D8) were performed in triplicate on an NRO aliquot representative of 10⁶ nuclei per reaction. To determine the purity of the NRO captured RNA relative DNA contamination, an RT minus reaction (sample B4) was included.

FIGS. 1a and 1b illustrate the amplification plot of the included samples (standard curve samples and NRO samples), the standard curve used to calculate mRNA concentrations, and the Table summarizing the data output from the Rotorgene instrument.

In this Example, the relative transcription level of the human BRN2 (FIG. 1a) and GAPDH endogenous (FIG. 1b) genes have been determine from NRO samples. Here, BRN2 is the target for quantification and GAPDH is the internal duplex control.

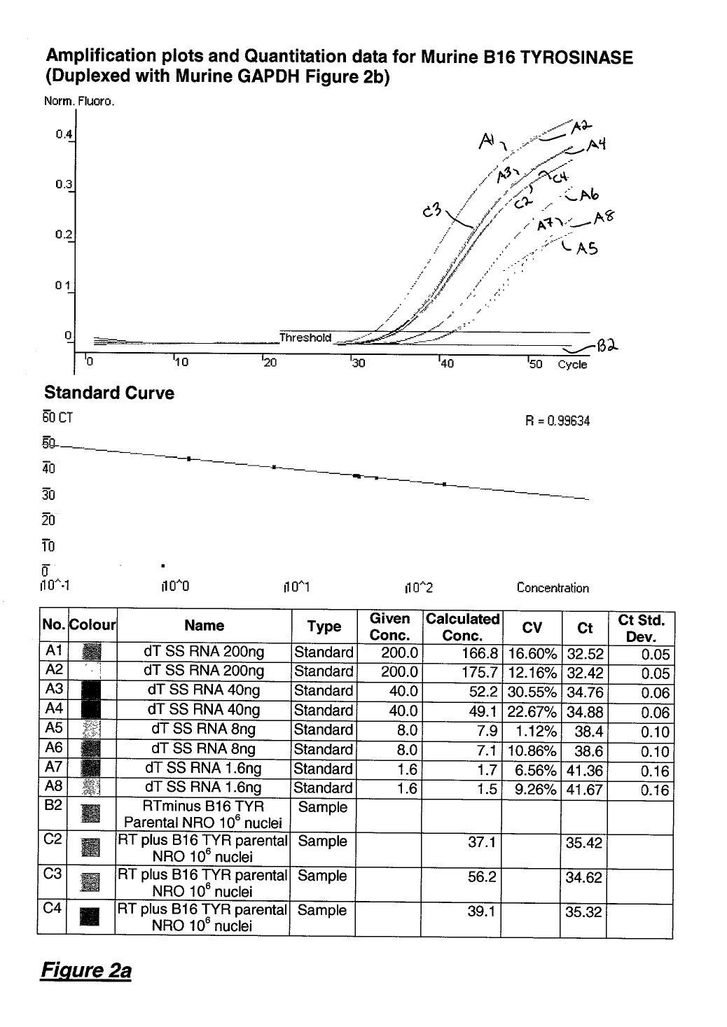

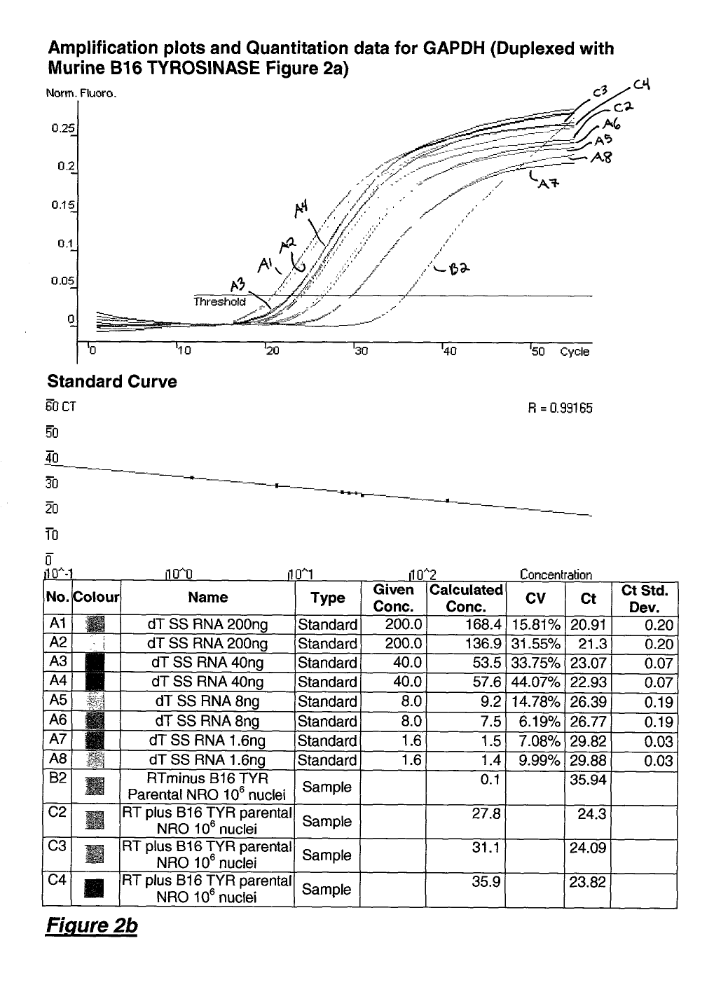

FIGS. 2a and 2b exemplify the establishment of a mRNA standard curve's (samples A1 to A8) for the parental non-transgenic murine cell line B16. To exemplify the quantitation of the template derived from a single NRO procedure, RT) positive reactions (samples C2-C4) were performed in triplicate on an NRO aliquot representative of 10⁶ nuclei per reaction. To determine the purity of the NRO captured RNA relative to DNA contamination, an RT minus reaction (sample B2) was included.

FIGS. 2a and 2b illustrate the amplification plot of the included samples (standard curve samples and NRO samples), the standard curve used to calculate mRNA concentrations, and the Table summarizing the data output from the Rotorgene instrument.

In this example, the relative transcription level of the murine tyrosinase (FIG. 2a) and GAPDH endogenous (FIG. 2b) genes have been determine from NRO samples. Here, tyrosinase gene is the target for quantification and GAPDH is the internal duplex control.

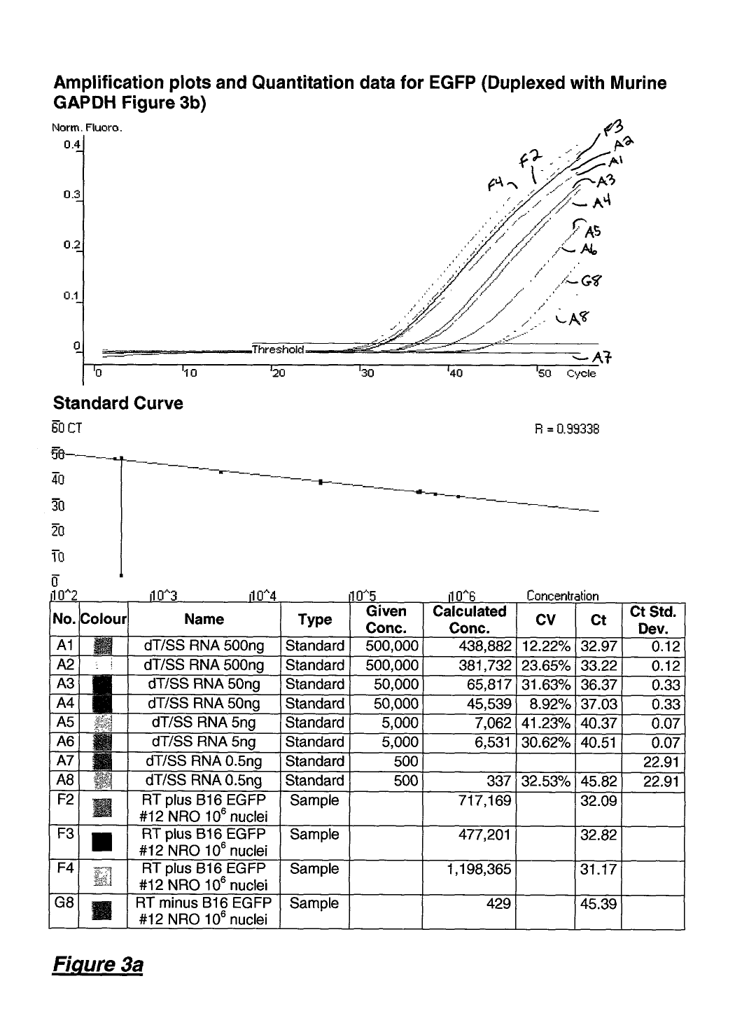

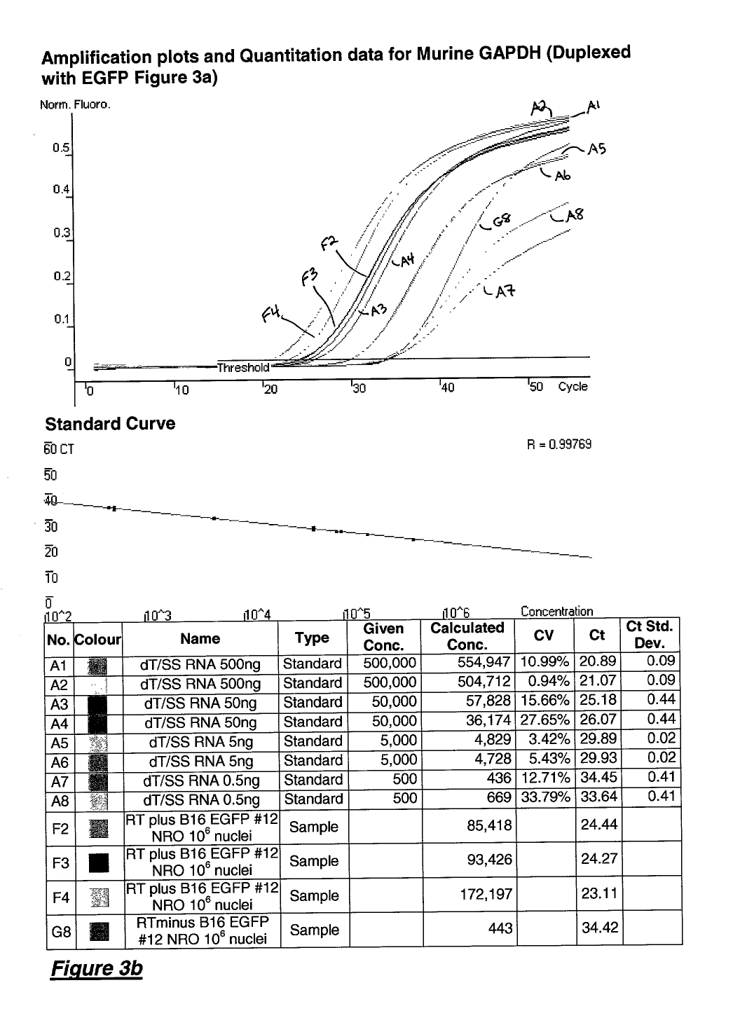

FIGS. 3a and 3b exemplify the establishment of a mRNA standard curve's (samples A1 to A8) for the EGFP transgenic murine cell line B16. To exemplify the quantitation of the template derived from a single NRO procedure, RT positive reactions (samples F2-F4) were performed in triplicate on an NRO aliquot representative of 10⁶ nuclei per reaction. To determine the purity of the NRO captured RNA relative to DNA contamination, an RT minus reaction (sample G8) was included.

FIGS. 3a and 3b illustrate the amplification plot of the included samples (standard curve samples and NRO samples), the standard curve used to calculate mRNA concentrations, and the Table summarizing the data output from the Rotorgene instrument.

In this Example, the relative transcription level of the exogenous transgene EGFP (FIG. 3a) derived from the plasmid pCMV.EGFP and the endogenous GAPDH gene (FIG. 3b) have been determined from NRO samples. Here, EGFP is the target for quantification and GAPDH is the internal duplex control.

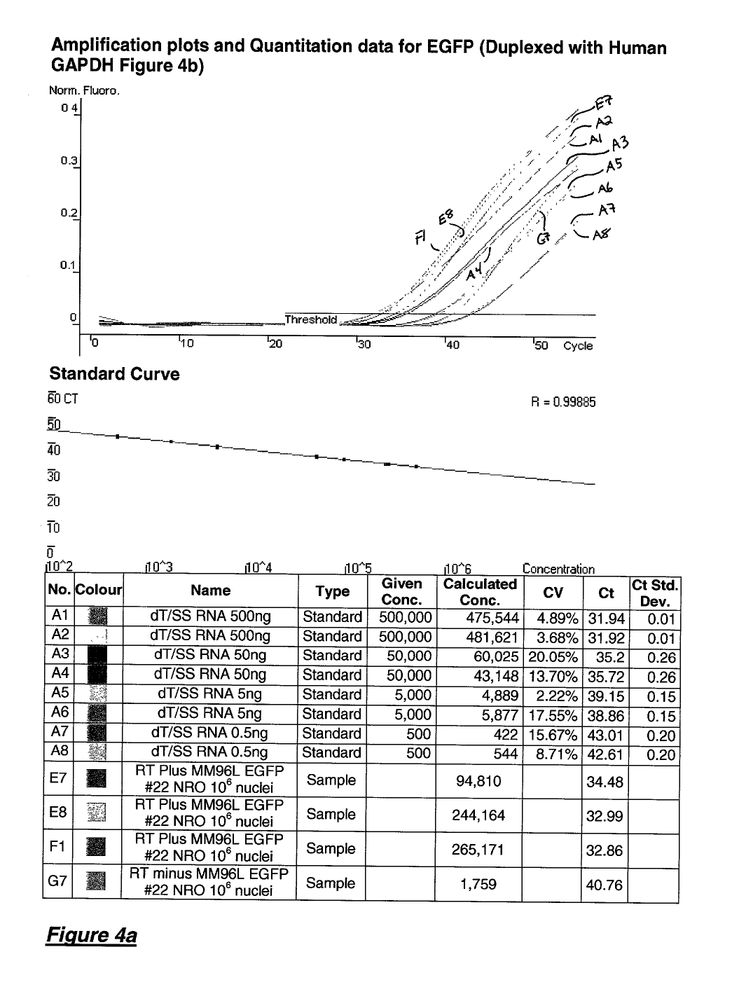

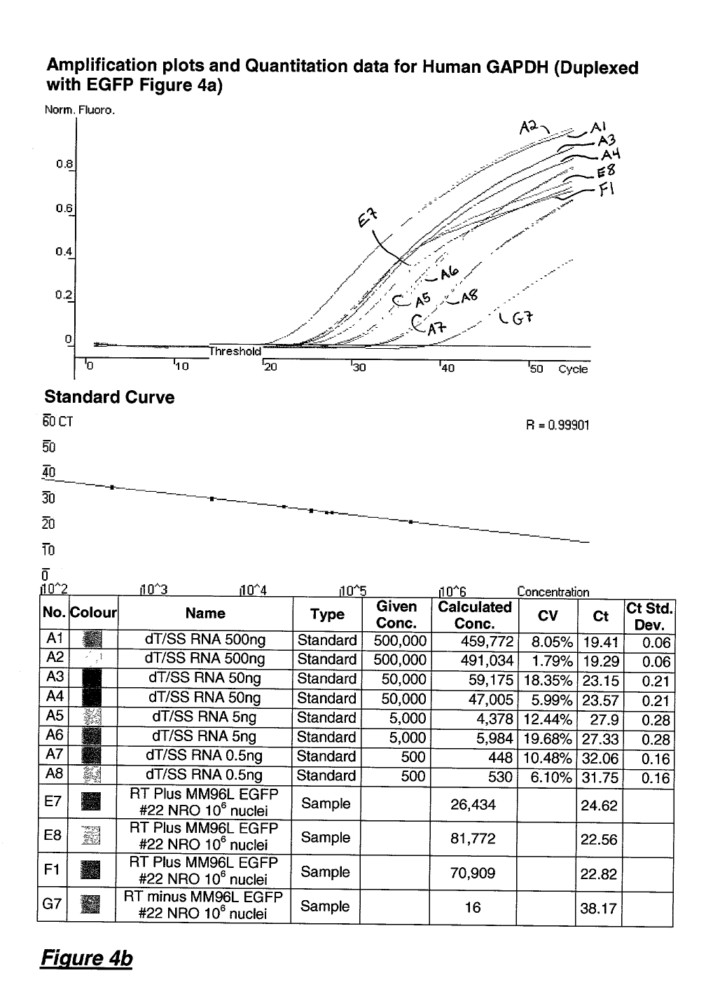

FIGS. 4a and 4b exemplify the establishment of a mRNA standard curve's (samples A1 to A8) for the EGFP transgenic human cell line MM96L. To exemplify the quantitation of the template derived from a single NRO procedure, RT positive reactions (samples E7-F1) were performed in triplicate on an NRO aliquot representative of 10⁶ nuclei per reaction. To determine the purity of the NRO captured RNA relative to DNA contamination, an RT minus reaction (sample G7) was included.

FIGS. 4a and 4b illustrate the amplification plot of the included samples (standard curve samples and NRO samples), the standard curve used to calculate mRNA concentrations, and the Table summarizing the data output from the Rotorgene instrument.

In this Example, the relative transcription level of the exogenous transgene EGFP (FIG. 4a) derived from the plasmid pCMV.EGFP and endogenous GAPDH gene (FIG. 4b) have been determine from NRO samples. Here, EGFP is the target for quantification and GAPDH is the internal duplex control.

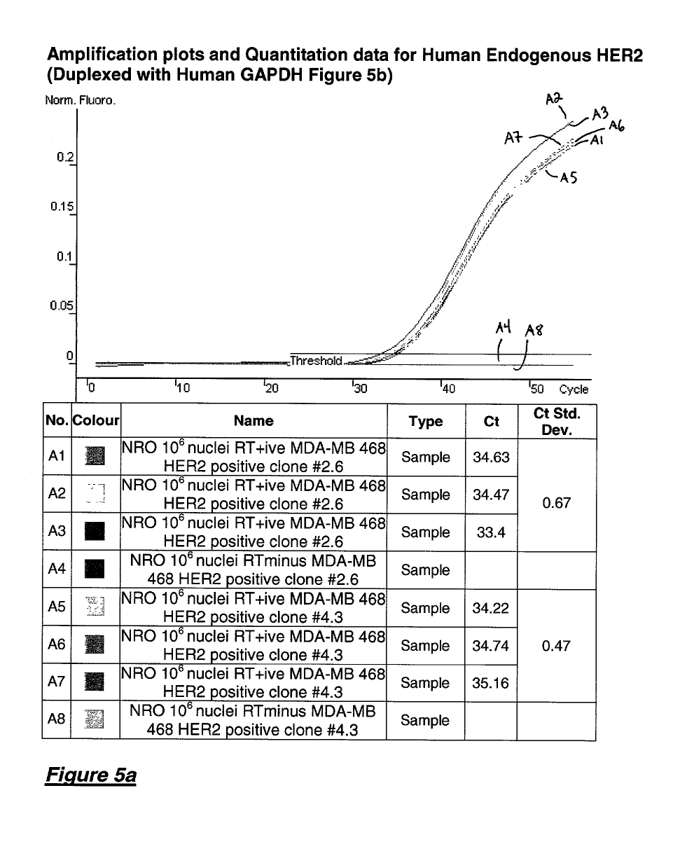

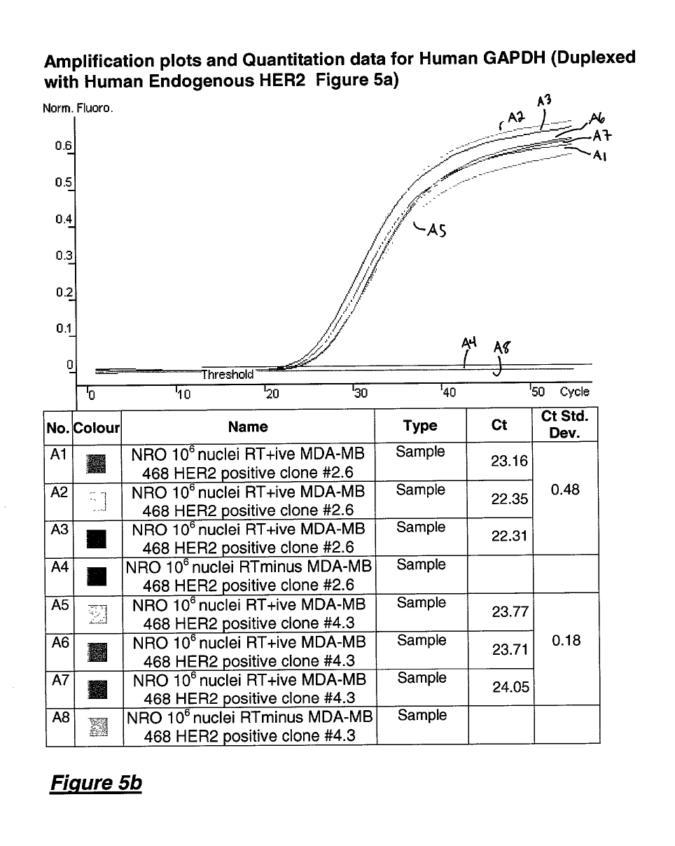

FIG. 5a and 5b exemplify the repeatability of the NRO method across two transgenic human cell lines, namely MDA-MB 468 clones #2.6 (samples A1 to A4) and #4.3 (samples A5 to A8). These clone where transfected with the plasmid pCMV.HER2.BGI2.2REH.

RT positive reactions (samples A1 to A3 and A5 to A7) were performed in triplicate on the two individual NRO preparations. A single RT minus reaction is included for each MDA-MB 468 clone (#2.6 sample A4; # 4.3 sample A8) to determine purity of NRO captured RNA relative to DNA contamination.

In this Example, the relative transcription level of the endogenous HER2 (FIG. 5a) and endogenous GAPDH gene (FIG. 5b) have been determine from NRO samples. Here, endogenous HER2 is the target for quantification and GAPDH is the internal duplex control.

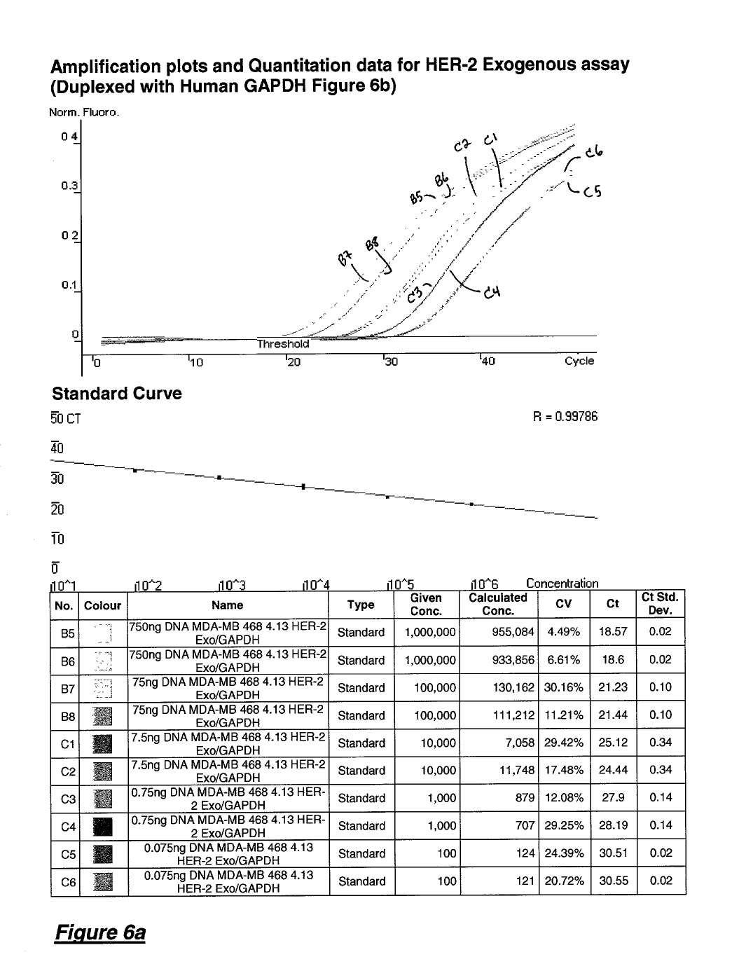

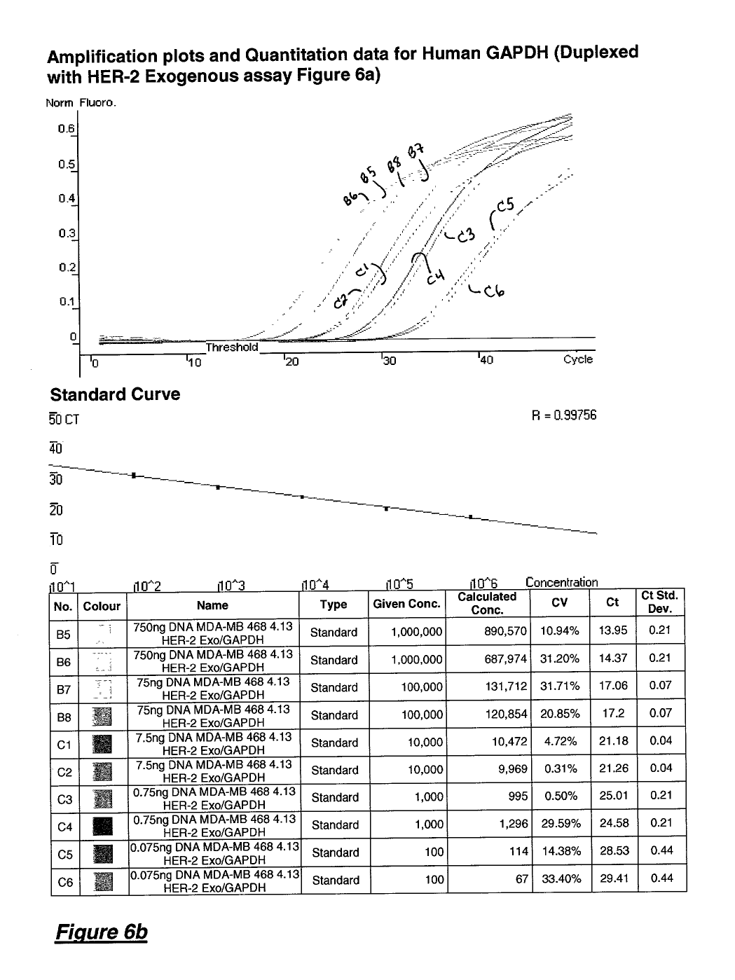

FIGS. 6a and 6b exemplify the linearity of the standard curves of the duplexed real-time PCR method on DNA template using the transgenic human cell lines MDA-MB 468 clone #4.13 (samples B5 to C6).

Genomic DNA was purified as described above and tritrated in 1/10 dilution across the range from 750 ng to 0.075 ng. Real-time PCR reactions were performed in duplicate. In this Example, the relative DNA concentration level of the exogenous transgene HER2.BGI2.2REH (FIG. 6a) and endogenous GAPDH gene (FIG. 6b) have been determine from NRO samples. Here, HER2.BGI2.2REH is the target for quantification and GAPDH is the internal duplex control. This latter experiment shows linearity of standard curve for DNA.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bassler et al., 1995, App. Environ. Microbiol. 61:_{—}3724-3728.

Belgrader et al, 1999, Science 284:449-450.

Dawson et al., 1989, Journal of Biological Chemistry 264:12830-12837.

Fahy et al., 1993, Nucleic Acids Research 21:1819-1826.

Hultman et al., 1989, Nucleic Acids Research 17:4937-4946.

Jeltsch et al., 1993, Analytical Biochemistry 209:278-283.

Kemp, D. J, 1992, Methods in Enzymology 216:116-126.

Kemp et al., 1989, Proc. Natl. Acad. Sci. USA 86:2423-2427.

Kohsaka et al., 1993, Nucleic Acids Research 21:3469-3472.

Lagerkvist et al., 1994, Proc. Natl. Acad. Sci. USA 91:2245-2249.

Lee et al., 1993, Nucleic Acids Res. 21:3761-3766.

Lew, A. M and Kemp, 1989, D. J, Nucleic Acids Research 17:5859.

Livak et al., 1995a, PCR Methods Applic. 4:357-362.

Livak et al., 1995b, Nature Genet. 9:341-342.

Lund et al., 1988, Nucleic Acids Research 16:10861-10880.

Olejnik et al., 1996, Nucleic Acids Research 24:361-366.

Stamm, S. and Brosius, J., 1991, Nucleic Acids Research 19:1350.

Syvanen et al., 1988, Nucleic Acids Research 16:11327-11338.

1. A method for determining the rate of transcription of a transcriptional unit in a composition of cells, said method comprising:—
lysing the cells and obtaining from the cells a preparation of nucleii comprising said transcriptional unit with nascent RNA strands attached thereto and placing same on ice to temporarily inhibit continued transcription and then placing said nucleii under conditions to permit transcription of the transcriptional unit in the presence of biotin-16-UTP to thereby provide a population of biotin-labeled nascent transcripts; and
isolating said biotin-labeled nascent transcripts by immobilizing same onto streptavidin-labeled iron beads and purifying same by magnetic separation and quantitatively determining the level of specific biotin-labeled RNA transcripts by subjecting the biotin-labeled RNA transcripts to real-time PCR.

2. The method of claim 1 wherein the cells are mammalian cells.

3. The method of claim 1 wherein the biotin-labeled RNA transcripts are eluted from the iron beads prior to the quantitative determination.

4. A kit for determining the rate of transcription of a transcriptional unit in a composition of cells, said kit comprising:
in compartmental form multiple compartments each adapted to comprise one or more of buffers, diluents and enzymes in single or multiple components which are required to be admixed prior to use, said kit further comprising instructions for use wherein the method is conducted by lysing the cells and obtaining from the cells a preparation of nucleii comprising said transcriptional unit with nascent RNA strands attached thereto and placing same on ice to temporarily inhibit continued transcription and then placing said said nucleii under conditions to permit transcription in the presence of biotin-16-UTP to thereby provide a population of biotin-labeled nascent transcripts; and
isolating said biotin-labeled nascent transcripts by immobilizing same onto streptavidin-labeled iron beads and purifying same by magnetic separation and quantitatively determining the level of specific biotin-labeled RNA transcripts by subjecting the biotin-labeled RNA transcripts to real-time PCR.

5. The method of claim 4 wherein the biotin-labeled RNA transcripts are eluted from the iron beads prior to the quantitative determination.


SEQUENCE LISTING (26 sequences)

SEQ ID NO: 1 - DNA, 18 bp, Unknown
  Primer
caaggctgtgggcaaggt

SEQ ID NO: 2 - DNA, 18 bp, Unknown
  Primer
ggaaggccatgccagtga

SEQ ID NO: 3 - DNA, 17 bp, Unknown
  Primer
caaggctgtgggcaagg

SEQ ID NO: 4 - DNA, 19 bp, Unknown
  Primer
cggaaggccatgccagtga

SEQ ID NO: 5 - DNA, 19 bp, Unknown
  Primer
gccttctgcccgaaaacac

SEQ ID NO: 6 - DNA, 19 bp, Unknown
  Primer
tgcggatgtcagccactgt

SEQ ID NO: 7 - DNA, 19 bp, Unknown
  Primer
gccttttgcccgaagacac

SEQ ID NO: 8 - DNA, 17 bp, Unknown
  Probe
atccctgagctgaacgg

SEQ ID NO: 9 - DNA, 17 bp, Unknown
  Probe
atcccagagctgaacgg

SEQ ID NO: 10 - DNA, 20 bp, Unknown
  Probe
tgggctatgcccgttcccgc

SEQ ID NO: 11 - DNA, 20 bp, Unknown
  Probe
tgggctatgcccgctcccgc

SEQ ID NO: 12 - DNA, 23 bp, Unknown
  Primer
gccgcgactctagatcataatca

SEQ ID NO: 13 - DNA, 24 bp, Unknown
  Primer
tgtgggaggttttttaaagcaagt

SEQ ID NO: 14 - DNA, 23 bp, Unknown
  Primer
gtactcgatttgtcctgccattg

SEQ ID NO: 15 - DNA, 21 bp, Unknown
  Primer
ggcatggacgagctgtacaag

SEQ ID NO: 16 - DNA, 20 bp, Unknown
  Primer
gtagaggtggcggagcatgt

SEQ ID NO: 17 - DNA, 20 bp, Unknown
  Probe
aaacctctacaaatgtggta

SEQ ID NO: 18 - DNA, 19 bp, Unknown
  Primer
ggctctgggcaccctgtat

SEQ ID NO: 19 - DNA, 18 bp, Unknown
  Primer
cagctgcagggcctcaaa

SEQ ID NO: 20 - DNA, 25 bp, unknown
  Primer
aactgtgacatttgcacagatgagt

SEQ ID NO: 21 - DNA, 23 bp, Unknown
  Primer
gaaggatgctgggctgagtaagt

SEQ ID NO: 22 - DNA, 28 bp, Unknown
  Primer
ggacctagtctctgccttctactctcta

SEQ ID NO: 23 - DNA, 17 bp, Unknown
  Primer
gcccctccccacactga

SEQ ID NO: 24 - DNA, 26 bp, Unknown
  Probe
caacgtgttctcgcagaccaccatct

SEQ ID NO: 25 - DNA, 26 bp, Unknown
  Probe
ttgggaggtcgtcaccctgaaaatcc

SEQ ID NO: 26 - DNA, 22 bp, Unknown
  Probe
ctggcccccctcagccctacaa